(19) United States
(12) Patent Application Publication
(10) Pub. No.: US 2010/0022799 A1
(43) Pub. Date: Jan. 28, 2010

(54) CARBONYLATION OF CONJUGATED DIENES

(75) Inventors: Graham Ronald Eastham, Redcar (GB); Mark Waugh, Redcar (GB); Philip Ian Richards, Redcar (GB)

Correspondence Address: VENABLE LLP, P.O. BOX 34385, WASHINGTON, DC, 20043-9998, US

(73) Assignee: LUCITE INTERNATIONAL UK LIMITED, Hampshire (GB)

(21) Appl. No.: 12/518,320

(22) PCT Filed: Dec. 20, 2007
(86) PCT No.: PCT/GB2007/050775

§ 371 Date: Jul. 9, 2009

(30) Foreign Application Priority Data
Dec. 21, 2006 (GB) 0625518.6

(51) Int. Cl. C07C 51/10

(52) U.S. Cl. 562/406

(57) ABSTRACT

A process for the carbonylation of a conjugated diene is described. The process comprises the steps of reacting a conjugated diene with carbon monoxide and a co-reactant having an active hydrogen in the presence of a solvent system and a catalyst system. The solvent system comprises a an aromatic carboxylic acid or, under some conditions, any carboxylic acid. The catalyst system is obtainable by combining: a. a metal of Group 8, 9 or 10 or a compound thereof: and b. a bidentate ligand of general formula (I) X¹(X²)-Q²-A-R—B-Q¹-X³(X⁴) (I) A and B each independently represent lower alkylene linking groups; R represents a cyclic hydrocarbyl structure to which Q¹ and Q² are linked, via the said linking group, on available adjacent cyclic atoms of the cyclic hydrocarbyl structure; the groups, X¹, X², X³ and X⁴ independently represent univalent radicals of up to 30 atoms having at least one tertiary carbon atom or X¹ and X² and/or X³ and X⁴ together form a bivalent radical of up to 40 atoms having at least two tertiary carbon atoms wherein each said univalent or bivalent radical is joined via said at least one or two tertiary carbon atoms respectively to the appropriate atom Q¹ or Q²; Q¹ and Q² each independently represent phosphorus, arsenic or 120 antimony; and, optionally, a source of anions. When the ratio of bidentate ligand: group 8, 9 or 10 metal is greater than 10:1 (mol:mol), the reaction proceeds with any carboxylic acid.

This invention relates to the carbonylation of conjugated dienes. Specifically, the invention relates to the carbonylation of a conjugated diene in the presence of an aromatic carboxylic acid.

The carbonylation of ethylenically unsaturated compounds using carbon monoxide in the presence of an alcohol or water and a catalyst system comprising a group 6, 8, 9 or 10 metal, for example, palladium, and a phosphine ligand, for example an alkyl phosphine, cycloalkyl phosphine, aryl phosphine, pyridyl phosphine or bidentate phosphine, has been described in numerous European patents and patent applications, for example EP-A-0055875, EP-A-04489472, EP-A-0106379, EP-A-0235864, EP-A-0274795, EP-A-0499329, EP-A-0386833, EP-A-0441447, EP-A-0489472, EP-A-0282142, EP-A-0227160, EP-A-0495547 and EP-A-0495548. In particular, EP-A-0227160, EP-A-0495547 and EP-A-0495548 disclose that bidentate phosphine ligands provide catalyst systems which enable high reaction rates to be achieved. C3 alkyl bridges between the phosphorus atoms are exemplified in EP0495548 together with tertiary butyl substituents on the phosphorus.

WO96/19434 subsequently disclosed that a particular group of bidentate phosphine compounds having an aryl bridge could provide remarkably stable catalysts which require little or no replenishment; that use of such bidentate catalysts leads to reaction rates which are significantly higher than those previously disclosed; and that little or no impurities are produced at high conversions.

WO 01/68583 discloses rates for the same process as WO 96/19434 when used for higher alkenes and when in the presence of an externally added aprotic solvent.

WO 98/42717 discloses a modification to the bidentate phosphines used in EP0495548 wherein one or both phosphorus atoms are incorporated into an optionally substituted 2-phospha-tricyclo[3.3.1.1{3,7}]decyl group or a derivative thereof in which one or more of the carbon atoms are replaced by heteroatoms (“2-PA” group). The examples include a number of alkoxycarbonylations of ethene, propene and some higher terminal and internal olefins.

WO 03/070370 extends the teaching of WO 98/42717 to bidentate phosphines having 1, 2 substituted aryl bridges of the type disclosed in WO96/19434. The suitable olefin substrates disclosed include several types having various substituents.

WO 04/103948 describes both the above types of ligand bridges as useful for 1,3-butadiene carbonylation and WO 05/082830 describes a selection of WO 04/103948 where the tertiary carbon substituents are different from each other on the respective phosphorus atoms.

WO 00/56695 relates to the use of phobane ligands for diene alkoxycarbonylation, optionally in the presence of benzoic acids as a source of anions. Hydroxycarbonylation is mentioned as a further possibility but is not exemplified; it is stated in this case that that the carbonylation product is used as the source of anions.

Surprisingly, it has now been discovered that remarkably high stability and/or reaction rate can be achieved with bidentate ligands by using a particular solvent system.

According to a first aspect of the present invention there is provided a process for the carbonylation of a conjugated diene comprising the steps of reacting said conjugated diene with carbon monoxide and a co-reactant having an active hydrogen in the presence of a solvent system comprising an aromatic carboxylic acid, a catalyst system and, optionally, a source of hydrogen, the catalyst system obtainable by combining:
(a) a metal of Group 8, 9 or 10 or a compound thereof: and
(b) a bidentate ligand of general formula (I)

X¹(X²)-Q²-A-R—B-Q¹-X³(X⁴) (I)

wherein:
A and B each independently represent lower alkylene linking groups;
R represents a cyclic hydrocarbyl structure to which Q¹ and Q² are linked, via the said linking group, on available adjacent cyclic atoms of the cyclic hydrocarbyl structure;
the groups X¹, X², X³ and X⁴ independently represent univalent radicals of up to 30 atoms having at least one tertiary carbon atom or X¹ and X² and/or X³ and X⁴ together form a bivalent radical of up to 40 atoms having at least two tertiary carbon atoms wherein each said univalent or bivalent radical is joined via said at least one or two tertiary carbon atoms respectively to the appropriate atom Q¹ or Q²;
Q¹ and Q² each independently represent phosphorus, arsenic or antimony; and optionally, a source or further source of anions.

According to a second aspect of the present invention there is provided a process for the carbonylation of a conjugated diene comprising the steps of reacting said conjugated diene with carbon monoxide and a co-reactant having an active hydrogen in the presence of a solvent system comprising a carboxylic acid, a catalyst system and, optionally, a source of hydrogen, the catalyst system obtainable by combining:
(a) a metal of Group 8, 9 or 10 or a compound thereof: and
(b) a bidentate ligand of general formula (I)

X¹(X²)-Q²-A-R—B-Q¹-X³(X⁴) (I)

wherein:
A and B each independently represent lower alkylene linking groups;
R represents a cyclic hydrocarbyl structure to which Q¹ and Q² are linked, via the said linking group, on available adjacent cyclic atoms of the cyclic hydrocarbyl structure; the groups X¹, X², X³ and X⁴ independently represent univalent radicals of up to 30 atoms having at least one tertiary carbon atom or X¹ and X² and/or X³ and X⁴ together form a bivalent radical of up to 40 atoms having at least two tertiary carbon atoms wherein each said univalent or bivalent radical is joined via said at least one or two tertiary carbon atoms respectively to the appropriate atom Q¹ or Q²;
Q¹ and Q² each independently represent phosphorus, arsenic or antimony; and, optionally, a source of anions;
wherein the ratio of bidentate ligand:group 8, 9 or 10 metal is greater than 10:1 (mol:mol).

In a continuous reaction, it is preferred to add a large excess of ligand at the start of the reaction and then feed a less than 10:1 ligand:metal ratio into the reactor until the ligand ratio falls below a pre-determined lower level (above 10:1) at which point the ligand ratio can be topped up again to a pre-determined upper level. To monitor this process the metal level may be determined using ICP MS using standards and the ligand concentration may be determined using GC, again using standards. Typical feed rates may be approximately 1:1.

Particularly preferred is when the ratio of bidentate ligand:group 8, 9 or 10 metal is greater than 20:1 (mol:mol), more preferably, 30:1 (mol:mol), most preferably 40:1(mol:mol). A typical range is greater than 10:1 to 1000:1, for example 20:1 to 500:1.

For the avoidance of doubt, references to Group 8, 9 or 10 metals herein should be taken to include Groups 8, 9 and 10 in the modern periodic table nomenclature. By the term “Group 8, 9 or 10” we preferably select metals such as Ru, Rh, Os, Ir, Pt and Pd. Preferably, the metals are selected from Ru, Pt and Pd. More preferably, the metal is Pd.

The ratio (v/v) of diene and co-reactant in the feed can vary between wide limits and suitably lies in the range of 10:1 to 1:500.

The co-reactant of the present invention may be any compound having a mobile hydrogen atom, and capable of reacting as a nucleophile with the diene under catalytic conditions. The chemical nature of the co-reactant determines the type of product formed. An especially advantageous co reactant is water so that hydroxcarbonylation is especially preferred. However, other co-reactants are also possible and may be advantageous such as a carboxylic acid, alcohol, ammonia or an amine, a thiol, or a combination thereof. If the co-reactant is water, the product obtained will be an unsaturated carboxylic acid. In the case of carboxylic acids the product is an unsaturated anhydride. For an alcohol co reactant, the product of the carbonylation is an ester.

Similarly, the use of ammonia (NH3) or a primary or secondary amine R⁸¹NH₂ or R⁸²R⁸³NH will produce an amide, and the use of a thiol R⁸¹SH will produce a thioester. In the above-defined coreactants, R⁸¹ R⁸² and/or R³ represent alkyl, alkenyl or aryl groups which may be unsubstituted or may be substituted by one or more substituents selected from halo, cyano, nitro, OR¹⁹, OC(O)R²⁰, C(O)R²¹, C(O)OR²², NR²³R²⁴, C(O)NR²⁵R²⁶, SR²⁹, C(O)SR³⁰, C(S)NR²⁷R²⁸, aryl or Het, wherein R¹⁹ to R³⁰ are defined herein, and/or be interrupted by one or more oxygen or sulphur atoms, or by silano or dialkylsilicon groups.

If ammonia or amines are employed, a small portion of co-reactants will react with acid present in the reaction to form an amide and water. Therefore, in the case of ammonia or amine-co-reactants, water is present.

Preferably the carboxylic acid co-reactant has the same number of carbon atoms as the diene reactant, plus one.

Preferred amine co-reactants have from 1 to 22, more preferably having 1 to 8 carbon atoms per molecule, and diamine co-reactants—having 2-22, more preferably 2 to 10 carbon atoms per molecule. The amines can be cyclic, part-cyclic, acylic, saturated or unsaturated (including aromatic), unsubstituted or substituted by one or more substituents selected from halo, cyano, nitro, OR¹⁹, C(O)R²⁰, C(O)R²¹, C(O)OR²², NR²³R²⁴, C(O)NR²⁵R²⁶, SR²⁹, C(O)SR³⁰, C(S)NR²⁷R²⁸, aryl, alkyl, Het, wherein R¹⁹ to R³⁰ are as defined herein and/or be interrupted by one or more (preferably less than a total of 4) oxygen, nitrogen, sulphur, silicon atoms or by silano or dialkyl silicon groups or mixtures thereof.

The thiol co-reactants can be cyclic, part-cyclic, acylic, saturated or unsaturated (including aromatic), unsubstituted or substituted by one or more substituents selected from halo, cyano, nitro, OR¹⁹, OC(O)R²⁰, C(O)R²¹, C(O)OR²², NR²³R²⁴, C(O)NR²⁵R²⁶, SR²⁹, C(O)SR³⁰, C(S)NR²⁷R²⁸, aryl, alkyl, Het, wherein R¹⁹ to R³⁰ are as defined herein and/or be interrupted by one or more (preferably less than a total of 4) oxygen, nitrogen, sulphur, silicon atoms or by silano or dialkyl silicon groups or mixtures thereof. Preferred thiol co-reactants are aliphatic thiols with 1 to 22, more preferably with 1 to 8 carbon atoms per molecule, and aliphatic dithiols with 2-22, more preferably 2 to 8 carbon atoms per molecule.

If a co-reactant should react with the acid serving as source of anions, then the amount of the acid to co-reactant should be chosen such that a suitable amount of free acid is present. Generally, a large surplus of acid over the co-reactant is preferred due to the enhanced-reaction rates.

As mentioned above, the present invention provides a process for the carbonylation of ethylenically unsaturated compound comprising contacting an ethylenically unsaturated compound with carbon monoxide and a co-reactant. The co-reactant is preferably a source of hydroxyl groups such as water, as mentioned above, or an alkanol in the presence of a catalyst compound as defined in the present invention.

Suitably, the source of hydroxyl groups includes an organic molecule having an hydroxyl functional group. Preferably, the organic molecule having a hydroxyl functional group may be branched or linear, and comprises an alkanol, particularly a C₁-C₃₀ alkanol, including aryl alkanols, which may be optionally substituted with one or more substituents selected from alkyl, aryl, Het, halo, cyano, nitro, OR¹⁹, OC(O)R²⁰, C(O)R²¹, C(O)OR²², NR²³R²⁴, C(O)NR²⁵R²⁶, C(S)R²⁷R²⁸, SR²⁹ or C(O)SR³⁰ as defined herein. Highly preferred alkanols are C₁-C₈ alkanols such as methanol, ethanol, propanol, iso-propanol, iso-butanol, t-butyl alcohol, n-butanol, phenol and chlorocapryl alcohol. Although the monoalkanols are most preferred, poly-alkanols, preferably, selected from di-octa ols such as diols, triols, tetra-ols and sugars may also be utilised. Typically, such polyalkanols are selected from 1,2-ethanediol, 1,3-propanediol, glycerol, 1,2,4 butanetriol, 2-(hydroxymethyl)-1,3-propanediol, 1,2,6 trihydroxyhexane, pentaerythritol, 1,1,1 tri(hydroxymethyl)ethane, nannose, sorbase, galactose and other sugars. Preferred sugars include sucrose, fructose and glucose. Especially preferred alkanols are methanol and ethanol. The most preferred alkanol is methanol.

The amount of alcohol is not critical. Generally, amounts are used in excess of the amount of substrate to be carbonylated. Thus the alcohol may serve as the reaction solvent as well, although, if desired, separate solvents may also be used.

It will be appreciated that the end product of the reaction is determined at least in part by the source of alkanol used. For instance, use of methanol produces the corresponding methyl ester. Conversely, use of water produces the corresponding acids. Accordingly, the invention provides a convenient way of adding the group —C(O)O C₁-C₃₀ alkyl or aryl or —C(O)OH across the ethylenically unsaturated bond.

Conjugated dienes contain at least two conjugated double bonds in the molecule. By conjugation is meant that the location of the 7c-orbital is such that it can overlap other orbitals in the molecule. Thus, the effects of compounds with at least two conjugated double bonds are often different in several ways from those of compounds with no conjugated bonds.

The conjugated diene preferably is a conjugated diene having from 4 to 22, more preferably from 4 to 10 carbon atoms per molecule. The conjugated diene can be substituted with one or more further substituents selected from aryl, alkyl, hetero (preferably oxygen), Het, halo, cyano, nitro, —OR¹⁹, —OC(O)R²⁰, R²¹, —C(O)R²², —C(O)OR²², —N(R²³)R²⁴, —C(O)N(R²⁵)R²⁶, —SR²⁹, —C(O)SR³⁰, —C(S)N(R²⁷)R²⁸ or —CF₃ wherein R¹⁹-R²⁸ are as defined herein or non-substituted. Most preferably, the conjugated diene is selected from conjugated pentadienes, conjugated hexadienes, cyclopentadiene and cyclohexadiene all of which may be substituted as set out above or unsubstituted. Especially preferred are 1,3-butadiene and 2-methyl-1,3-butadiene and most especially preferred is non-substituted 1,3-butadiene.

The person with average skill in the art will further realise that the process of the present invention can also be used to prepare carboxylic mono-acids and/or carboxylic diacids. Carboxylic mono-acids and/or carboxylic diacids are prepared by reacting conjugated dienes with carbon monoxide and using water as a hydroxyl group containing compound. In this case, the carbonylation product, i.e. the carboxylic acid or di-acid can be used as an additional source of anions.

The aromatic carboxylic acid used in the carbonylation reaction such as a hydroxycarbonylation reaction is preferably any optionally substituted C₁-C₃₀ aromatic compound such as those based on phenyl, napthyl, cyclopentadienyl anion(s), indenyl, pyridinyl, and pyrollyl groups and having at least one carboxylic acid group associated with the aromatic ring, more preferably any C₁ to C₁₆ aromatic compound having at least one carboxylic acid group. The pKa of the acid is preferably greater than about 2 measured in dilute aqueous solution at 18° C. The pKa is preferably less than about 6 measured in dilute aqueous solution at 18° C., more preferably, less than 5.

The carboxylic acid group means a —COOH group and this may be attached directly to a cyclic ring atom of the aromatic ring but may also be attached to an α or β carbon to the ring, more preferably either attached to an α carbon or directly to the ring, most preferably, attached directly to the ring.

The aromatic compound may be substituted with one or more of the following: alkyl groups; aryl groups; hydroxy groups; alkoxy groups such as, for example, methoxy; amino groups or halo groups such as, for example F, Cl, I and Br.

The aromatic ring of the carboxylic acid may substituted on any available carbon atom. Preferably, the aromatic ring is mono- or di-substituted. Examples of suitable aromatic carboxylic acids include benzoic acids; naphthoic acids; and cyclopentadienyl acids, particularly preferred are substituted aromatic acids, including for example, C₁-C₄ alkyl substituted benzoic acids, such as 2,4,6-trimethyl benzoic acid, or 2,6-dimethyl benzoic acid and O-toluic acid (2-methyl benzoic acid), 2-nitrobenzoic acid, 6-chloro-2-methylolbenzoic acid, 4-aminobenzoic acid, 2-chloro-6-hydroxybenzoic acid, 2-cyanobenzoic acid, 3-cyanobenzoic acid, 4-cyanobenzoic acid 2,4-dihydroxybenzoic, 3-nitrobenzoic acid, 2-phenylbenzoic acid, 2-tert-butylbenzoic acid, 2-napthoic acid, 1-napthoic acid, 2,4-dimethylbenzoic acid, 3-methylbenzoic acid, 3,5-dimethylbenzoic acid, 4-hydroxybenzoic acid, 2-fluorobenzoic acid, 3-propoxybenzoic acid, 3-ethoxybenzoic acid, 2-propoxybenzoic acid, 2,2-diphenylpropionic acid, 2-meyhoxyphenylacetic acid, ortho-anisic acid, meta-anisic acid, 4-tert-butylbenzoic acid and 2-ethoxybenzoic acid.

Preferably, the aromatic carboxylic acid is substituted by only one group in addition to the group bearing the carboxylic acid. Preferably, an alkyl group substitutes the aromatic ring of the carboxylic acid. An especially preferred compound is O-toluic acid.

The carboxylic acid used in the second aspect of the present invention may be any optionally substituted C₁-C₃₀ compound having, in addition, at least one carboxylic acid group, more preferably any C₁ to C₁₆ compound having at least one carboxylic acid group. The pKa of the acid is preferably greater than about 2 measured in dilute aqueous solution at 18° C. The pKa is preferably less than about 6 measured in dilute aqueous solution at 18° C. Examples of suitable carboxylic acids include: optionally substituted C₁-C₁₂alkanoic acids such as acetic acid, propionic acids, butyric acids, pentanoic acids, hexanoic acids, nonanoic acids; C₁-C₁₂ alkenoic acids such as propenoic acids such as acrylic acid, butenoic acids such as methacrylic acid, pentenoic acids, hexenoic acids and heptenoic acids; lactic acid; which may all where possible be linear or branched, cyclic, part cyclic, or acyclic and apart from that they may be interrupted with hetero atoms may be unsubstituted or substituted with one or more further substituents selected from aryl, alkyl, hetero (preferably oxygen), Het, halo, cyano, nitro, —OR¹⁹, —OC(O)R²⁰, —C(O)R²¹, —C(O)OR²², —N(R²³)R²⁴, —C(O)N(R²⁵)R²⁶, —SR²⁹, —C(O)SR³⁰, —C(S)N(R²⁷)R²⁸ or —CF₃ wherein R¹⁹-R²⁸ are as defined herein; and aromatic carboxylic acids such as those described above.

A particularly preferred carboxylic acid is the acid product of the carbonylation reaction.

In the carbonylation reaction such as a hydroxycarbonylation reaction of the first aspect of the invention, preferably, the ratio of equivalents of bidentate ligand to group 8, 9 or 10 metal is at least 1:1 mol/mol. Preferably, the ligand is in excess of metal mol/mol. Preferably, the ratio of equivalents of bidentate ligand:group 8, 9 or 10 metal is greater than 1:1, preferably, greater than 4:1, more preferably, greater than 10:1.

Preferably, the solvent system comprises a carboxylic acid as defined above (preferably an aromatic carboxylic acid) and at least one co-solvent.

Suitable co-solvents for use in the present invention include ketones, such as for example methylbutylketone; ethers, such as for example anisole (methyl phenyl ether), 2,5,8-trioxanonane (diglyme), diethyl ether, dimethyl ether, methyl-tert-butylether (MTBE), tetrahydrofuran, diphenylether, diisopropylether and the dimethylether of di-ethylene-glycol; oxanes, such as for example dioxane; esters, such as for example methylacetate, dimethyladipate methyl benzoate, dimethyl phthalate and butyrolactone; amides, such as for example dimethylacetamide, N-methylpyrrolidone and dimethyl formamide; sulfoxides and sulphones, such as for example dimethylsulphoxide, di-isopropylsulphone, sulfolane (tetrahydrothiophene-2,2-dioxide), 2-methylsulfolane, diethyl sulphone, tetrahydrothiophene 1,1-dioxide and 2-methyl-4-ethylsulfolane; aromatic compounds, including halo variants of such compounds e.g. benzene, toluene, ethyl benzene o-xylene, m-xylene, p-xylene, chlorobenzene, o-dichlorobenzene, m-dichlorobenzene: alkanes, including halo variants of such compounds e.g. hexane, heptane, 2,2,3-trimethylpentane, methylene chloride and carbon tetrachloride; nitriles e.g. benzonitrile and acetonitrile.

Alternatively, the co-solvent can be any one or more further carboxylic acid such as any of those carboxylic acids mentioned above.

Very suitable are aprotic co-solvents having a dielectric constant that is below a value of 50, more preferably 1-30, most preferably, 1-10, especially in the range of 2 to 8, at 298 or 293K and 1×10⁵Nm 2. In the context herein the dielectric constant for a given co-solvent is used in its normal meaning of representing the ratio of the capacity of a condenser with that substance as dielectric to the capacity of the same condenser with a vacuum for dielectric. Values for the dielectric constants of common organic liquids can be found in general reference books, such as the Handbook of Chemistry and Physics, 76^{th} edition, edited by David R. Lide et al, and published by CRC press in 1995, and are usually quoted for a temperature of about 20° C. or 25° C., i.e. about 293.15 k or 298.15 K, and atmospheric pressure, i.e. about 1×10⁵Nm⁻², and can readily be converted to 298.15 K and atmospheric pressure using the conversion factors quoted. If no literature data for a particular compound is available, the dielectric constant may be readily measured using established physico-chemical methods.

Measurement of a dielectric constant of a liquid can easily be performed by various sensors, such as immersion probes, flow-through probes, and cup-type probes, attached to various meters, such as those available from the Brookhaven Instruments Corporation of Holtsville, N.Y. (e.g., model BI-870) and the Scientifica Company of Princeton, N.J. (e.g. models 850 and 870). For consistency of comparison, preferably all measurements for a particular filter system are performed at substantially the same sample temperature, e.g., by use of a water bath. Generally, the measured dielectric constant of a substance will increase at lower temperatures and decrease at higher temperatures. The dielectric constants falling within any ranges herein, may be determined in accordance with ASTM D924

However, if there is doubt as to which technique to use to determine the dielectric constant a Scientifica Model 870 Dielectric Constant Meter with a 1-200 ∈ range setting should be used.

For example, the dielectric constant of methyl-tert-butyl ether is 4.34 (at 293 K), of dioxane is 2.21 (at 298 K), of toluene is 2.38 (at 298 K), tetrahydrofuran is 7.5 (at 295.2 K) and of acetonitrile is 37.5 (at 298 K). The dielectric values are taken from the handbook of chemistry and physics and the temperature of the measurement is given.

Alternatively, the reaction may proceed in the absence of an aprotic co-solvent not generated by the reaction itself.

Alternatively, a protic co-solvent may be used. The protic co-solvent may include a further carboxylic acid or an alcohol. Suitable protic co-solvents include the conventional protic solvents known to the person skilled in the art, such as water, lower alcohols, such as, for example, methanol, ethanol and isopropanol, and primary and secondary amines. Mixtures of the aprotic and protic co-solvents may also be employed.

By protic co-solvent is meant any solvent that carries a donatable hydrogen ion such as those attached to oxygen as in a hydroxyl group or nitrogen as in a amine group. By aprotic co-solvent is meant a type of solvent which neither donates nor accepts protons.

In the process according to the present invention, the carbon monoxide may be used in pure form or diluted with an inert gas such as nitrogen, carbon dioxide or a noble gas such as argon.

Hydrogen may optionally be added to the carbonylation reaction to improve reaction rate. Suitable levels of hydrogen when utilised may be in the ratio of between 0.1 and 20% vol/vol of the carbon monoxide, more preferably, 1-20% vol/vol of the carbon monoxide, more preferably, 2-15% vol/vol of the carbon monoxide, most preferably 3-10% vol/vol of carbon monoxide.

Hydrogen, if present, is preferably present at a partial pressure of between 1×10 and 20×10 Pa, preferably between 2×10⁵ and 10×10⁵ Pa, and most preferably, at a partial pressure of about 5×10⁵ Pa.

The molar ratio of the amount of conjugated diene, especially 1,3-butadiene used in the reaction to the amount of carboxylic acid is not critical and may vary between wide limits, e.g. from 0.001:1 to 100:1 mol/mol. Preferably, the molar ratio of conjugated diene, especially 1,3-butadiene to carboxylic acid is selected to minimise the diene concentration such that it reacts preferentially to form the corresponding acid, in the case of 1,3-butadiene, pentenoic acids. Typically, in a process of the invention, particularly a continuous process, the molar ratio is between 1:1 and 70:1, more preferably, 1:1 to 50:1.

The amount of the catalyst of the invention used in the carbonylation reaction such as a hydroxycarbonylation reaction is not critical. Good results may be obtained, preferably when the amount of Group 8, 9 or 10 metal is in the range 10⁻⁷ to 10⁻¹ moles per mole of conjugated diene, especially 1,3-butadiene, more preferably, 10⁻⁶ to 10⁻² moles, most preferably 10⁻⁵ to 10⁻² moles per mole of conjugated diene. Preferably, the amount of bidentate compound of formula I to conjugated diene is in the range 10⁻⁷ to 10⁻¹, more preferably, 10⁻⁶ to 10⁻², most preferably, 10⁻⁵ to 10⁻² moles per mole of conjugated diene. Preferably, the amount of catalyst is sufficient to produce product at an acceptable rate commercially.

Preferably, the carbonylation is carried out at temperatures of between −30 to 170° C., more preferably −10° C. to 160° C., most preferably 20° C. to 150° C. An especially preferred temperature is one chosen between 40° C. to 150° C. Alternatively, the carbonylation can be carried out at moderate temperatures, it is particularly advantageous to be able to carry out the reaction at room temperature (20° C.).

Preferably, when operating a low temperature carbonylation, the carbonylation is carried out between −30° C. to 49° C., more preferably, −10° C. to 45° C., still more preferably 0° C. to 45° C., most preferably 10° C. to 45° C. Especially preferred is a range of 10 to 35° C.

Preferably, the carbonylation is carried out at a CO partial pressure of between 1×10 N.m⁻²-120×10⁵ N.m⁻², more preferably 10×10 N.m⁻²-100×10⁵N.m⁻², most preferably 20-90×10⁵ N.m⁻². Especially preferred is a CO partial pressure of 40 to 80×10⁵ N.m⁻².

The cyclic hydrocarbyl structure which R represents may be aromatic, non-aromatic, mixed aromatic and non-aromatic, mono-, bi-, tri- or polycyclic, bridged or unbridged, substituted or unsubstituted or interrupted by one or more hetero atoms, with the proviso that the majority of the cyclic atoms (ie more than half) in the structure are carbon. The available adjacent cyclic atoms to which the Q¹ and Q² atoms are linked to form part of at least one ring. This ring to which the Q¹ and Q² atoms are immediately linked via the linking group may itself be an aromatic or non-aromatic ring. When the ring to which the Q¹ and Q² atoms are directly attached via the linking group is non-aromatic, any further rings in a bicyclic, tricyclic or polycyclic structure can be aromatic or non-aromatic or a combination thereof. Similarly, when the ring to which the Q¹ and Q² atoms are immediately attached via the linking group is aromatic, any further rings in the hydrocarbyl structure may be non-aromatic or aromatic or a combination thereof.

For simplicity, these two types of bridging group R will be referred to as an aromatic bridged cyclic hydrocarbyl structure or a non-aromatic bridged cyclic hydrocarbyl structure irrespective of the nature of any further rings joined to the at least one ring to which the Q¹ and Q² atoms are linked via the linking groups directly.

The non-aromatic bridged cyclic hydrocarbyl structure which is substituted by A and B at adjacent positions on the at least one non-aromatic ring preferably, has a cis-conformation with respect to the A and B substituents i.e. A and B extend away from the structure on the same side thereof.

Preferably, the non-aromatic bridged cyclic hydrocarbyl structure has from 3 up to 30 cyclic atoms, more preferably from 4 up to 18 cyclic atoms, most preferably from 4 up to 12 cyclic atoms and especially 5 to 8 cyclic atoms and may be monocyclic or polycyclic. The cyclic atoms may be carbon or hetero, wherein references to hetero herein are references to sulphur, oxygen and/or nitrogen. Typically, the non-aromatic bridged cyclic hydrocarbyl structure has from 2 up to 30 cyclic carbon atoms, more preferably from 3 up to 18 cyclic carbon atoms, most preferably from 3 up to 12 cyclic carbon atoms and especially 3 to 8 cyclic carbon atoms, may be monocyclic or polycyclic and may or may not be interrupted by one or more hetero atoms. Typically, when the non-aromatic bridged cyclic hydrocarbyl structure is polycylic it is preferably bicyclic or tricyclic. The non-aromatic bridged cyclic hydrocarbyl structure as defined herein may include unsaturated bonds. By cyclic atom is meant an atom which forms part of a cyclic skeleton.

The non-aromatic bridged cyclic hydrocarbyl structure, apart from that it may be interrupted with hetero atoms may be unsubstituted or substituted with one or more further substituents selected from aryl, alkyl, hetero (preferably oxygen), Het, halo, cyano, nitro, —OR¹⁹, —OC(O)R²⁰, —C(O)R²¹, —C(O)OR²², —N(R²³)R²⁴, —C(O)N(R²⁵)R²⁶, —SR²⁹, —C(O)SR³⁰, —C(S)N(R²⁷)R²⁸ or —CF₃ wherein R¹⁹-R²⁸ are as defined herein.

The non-aromatic bridged cyclic hydrocarbyl structure may be selected from cyclohexyl, cyclopentyl, cyclobutyl, cyclopropyl, cycloheptyl, cyclooctyl, cyclononyl, tricyclodecyl, piperidinyl, morpholinyl, norbornyl, isonorbornyl, norbornenyl, isonorbornenyl, bicyclo[2,2,2]octyl, tetrahydrofuryl, dioxanyl, O-2,3-isopropylidene-2,3-dihydroxy-ethyl, cyclopentanonyl, cyclohexanonyl, cyclopentenyl, cyclohexenyl, cyclohexadienyl, cyclobutenyl, cyclopentenonyl, cyclohexenonyl, adamantyl, furans, pyrans, 1,3 dioxane, 1,4 dioxane, oxocene, 7-oxabicyclo[2.2.1]heptane, pentamethylene sulphide, 1,3 dithiane, 1,4 dithiane, furanone, lactone, butyrolactone, pyrone, succinic anhydride, cis and trans 1,2-cyclohexanedicarboxylic anhydride, glutaric anhydride, pyrollidine, piperazine, imidazole, 1,4,7 triazacyclononane, 1,5,9 triazacyclodecane, thiomorpholine, thiazolidine, 4,5-diphenyl-cyclohexyl, 4 or 5-phenyl-cyclohexyl, 4,5-dimethyl-cyclohexyl, 4 or 5-methylcyclohexyl, 1,2-decalinyl, 2,3,3a,4,5,6,7,7a-octahydro-1H-inden-5,6-yl, 3a, 4, 5, 6, 7, 7a-hexahydro-1H-inden-5,6-yl, 1, 2 or 3 methyl-3a,4,5,6,7,7a hexahydro-1H-inden-5,6-yl, trimethylene norbornanyl, 3a, 4,7,7a-tetrahydro-1H-inden-5,6-yl, 1, 2 or 3-dimethyl-3a, 4,5,6,7,7a-hexahydro-1H-inden 5,6-yls, 1,3-bis(trimethylsilyl)-3a,4,5,6,7,7a-hexahydro-3H-isobenzofuran and wherein the linking group A or B is joined to available non-substituted adjacent cyclic atoms.

R may represent a non-aromatic bridged cyclic hydrocarbyl structure having at least one non-aromatic ring to which the Q¹ and Q² atoms are linked on available adjacent cyclic atoms of the at least one ring. Apart from that it may be in the form of a polycyclic structure, the non-aromatic bridged cyclic hydrocarbyl structure may be unsubstituted or substituted with at least one substituent, preferably on at least one further non-adjacent cyclic atom of the at least one ring.

By the term one further non-adjacent cyclic atom is meant any further cyclic atom in the ring which is not adjacent to any one of said available adjacent cyclic atoms to which the Q¹ and Q² atoms are linked.

However, the cyclic atoms adjacent to the said available adjacent cyclic atoms and cyclic atoms elsewhere in the hydrocarbyl structure may also be substituted suitable substituents for the cyclic atom(s) are defined herein.

For the avoidance of doubt, references to the cyclic atoms adjacent to the said available adjacent cyclic atoms or the like is not intended to refer to one of the said two available adjacent cyclic atoms themselves. As an example, a cyclohexyl ring joined to a Q¹ atom via position 1 on the ring and joined to a Q² atom via position 2 on the ring has two said further non adjacent cyclic atoms as defined at ring position 4 and 5 and two adjacent cyclic atoms to the said available adjacent cyclic atoms at positions 3 and 6.

The term a non-aromatic bridged cyclic hydrocarbyl structure means that the at least one ring to which the Q¹ and Q² atom are linked via B & A respectively is non-aromatic, and aromatic should be interpreted broadly to include not only a phenyl type structure but other rings with aromaticity such as that found in the cyclopentadienyl anion ring of ferrocenyl, but, in any case, does not exclude aromatic substituents on this non-aromatic at least one ring.

The substituents on the said cyclic atoms of the non-aromatic bridged hydrocarbyl structure may be selected to encourage greater stability but not rigidity of conformation in the cyclic hydrocarbyl structure. The substituents may, therefore, be selected to be of the appropriate size to discourage or lower the rate of non-aromatic ring conformation changes. Such groups may be independently selected from lower alkyl, aryl, het, hetero, halo, cyano, nitro, —OR¹⁹, —OC(O)R²⁰, —C(O)R²¹, —C(O)OR²², —N(R²³)R²⁴, —C(O)N(R²⁵)R²⁶, —SR²⁹, —C(O)SR³⁰, —C(S)N(R²⁷)R²⁸ or —CF₃, more preferably, lower alkyl, or hetero most preferably, C₁-C₆ alkyl. Where there are two or more further cyclic atoms in the hydrocarbyl structure they may each be independently substituted as detailed herein. Accordingly, where two such cyclic atoms are substituted, the substituents may combine to form a further ring structure such as a 3-20 atom ring structure. Such a further ring structure may be saturated or unsaturated, unsubstituted or substituted by one or more substituents selected from halo, cyano, nitro, OR¹⁹, OC(O)R²⁰, C(O)R²¹, C(O)OR²², NR²³R²⁴, C(O)NR²⁵R²⁶, SR²⁹, C(O)SR³⁰, C(S)NR²⁷R²⁸, aryl, alkyl, Het, wherein R¹⁹ to R³⁰ are as defined herein and/or be interrupted by one or more (preferably less than a total of 4) oxygen, nitrogen, sulphur, silicon atoms or by silano or dialkyl silicon groups or mixtures thereof.

Particularly preferred substituents are methyl, ethyl, propyl, isopropyl, phenyl, oxo, hydroxy, mercapto, amino, cyano and carboxy. Particularly preferred substituents when two or more further non adjacent cyclic atoms are substituted are x,y-dimethyl, x,y-diethyl, x,y-dipropyl, x,y-di-isopropyl, x,y-diphenyl, x,y-methyl/ethyl, x,y-methyl/phenyl, saturated or unsaturated cyclopentyl, saturated or unsaturated cyclohexyl, 1,3 substituted or unsubstituted 1,3H-furyl, un-substituted cyclohexyl, x,y-oxo/ethyl, x,y-oxo/methyl, disubstitution at a single ring atom is also envisaged, typically, x,x-lower dialkyl. More typical substituents are methyl, ethyl, n-propyl, iso-propyl, n-butyl, isobutyl, t-butyl, or oxo, most typically methyl or ethyl, or oxo most typically, methyl; wherein x and y stand for available atom positions in the at least one ring.

Preferably, further substitution of said non-aromatic cyclic hydrocarbyl structure is not on said available adjacent carbon atoms to which said Q¹ and Q² atoms are linked. The non-aromatic cyclic hydrocarbyl structure may be substituted at one or more said further cyclic atoms of the hydrocarbyl structure but is preferably substituted at 1, 2, 3 or 4 such cyclic atoms, more preferably 1, 2 or 3, most preferably at 1 or 2 such cyclic atoms, preferably on the at least one non-aromatic ring. The substituted cyclic atoms may be carbon or hetero but are preferably carbon.

When there are two or more substituents on the said cyclic hydrocarbyl structure they may meet to form a further ring structure unless excluded herein.

The non-aromatic bridged cyclic hydrocarbyl structure may be selected from 4 and/or 5 lower alkylcyclohexane-1,2-diyl, 4 lower alkylcyclopentane-1,2-diyl, 4, 5 and/or 6 lower alkylcycloheptane-1,2-diyl, 4, 5, 6 and/or 7 lower alkylcyclooctane-1,2-diyl, 4, 5, 6, 7 and/or 8 lower alkylcyclononane-1,2-diyl, 5 and/or 6 lower alkyl piperidinane-2,3-diyl, 5 and/or 6 lower alkyl morpholinane-2,3-diyl, O-2,3-isopropylidene-2,3-dihydroxy-ethane-2,3-diyl, cyclopentan-one-3,4-diyl, cyclohexanone-3,4-diyl, 6-lower alkyl cyclohexanone-3,4-diyl, 1-lower alkyl cyclopentene-3,4-diyl, 1 and/or 6 lower alkyl cyclohexene-3,4-diyl, 2 and/or 3 lower alkyl cyclohexadiene-5,6-diyl, 5 lower alkyl cyclohexen-4-one-1,2-diyl, adamantyl-1-2-diyl, 5 and/or 6 lower alkyl tetrahydropyran-2,3 diyl, 6-lower alkyl dihydropyran-2,3 diyl, 2-lower alkyl 1,3 dioxane-5,6-diyl, 5 and/or 6 lower alkyl-1,4 dioxane-2,3-diyl, 2-lower alkyl pentamethylene sulphide 4,5-diyl, 2-lower alkyl-1,3 dithiane-5,6-diyl, 2 and/or 3-lower alkyl 1,4 dithiane-5,6-diyl, tetrahydro-furan-2-one-4,5-diyl, delta-valero lactone 4,5-diyl, gamma-butyrolactone 3,4-diyl, 2H-dihydropyrone 5,6-diyl, glutaric anhydride 3,4-diyl, 1-lower alkyl pyrollidine-3,4-diyl, 2,3 di-lower alkyl piperazine-5,6-diyl, 2-lower alkyl dihydro imidazole-4,5-diyl, 2,3,5 and/or 6 lower alkyl-1,4,7 triazacyclononane-8,9-diyl, 2,3,4 and/or 10 lower alkyl-1,5,9 triazacyclodecane 6,7-diyl, 2,3-di-lower alkyl thiomorpholine-5,6-diyl, 2-lower alkyl-thiazolidine-4,5-diyl, 4,5-diphenyl-cyclohexane-1,2-diyl, 4 and/or 5-phenyl-cyclohexane-1,2-diyl, 4,5-dimethyl-cyclohexane-1,2-diyl, 4 or 5-methylcyclohexane-1,2-diyl, 2, 3, 4 and/or 5 lower alkyl-decahydronaphthalene 8,9-diyl, bicyclo[4.3.0] nonane-3,4 diyl, 3a,4,5,6,7,7a-hexahydro-1H-inden-5,6-diyl, 1, 2 and/or 3 methyl-3a,4,5,6,7,7a hexahydro-1H-inden-5,6-diyl, Octahydro-4,7 methano-indene-1,2-diyl, 3a,4,7,7a-tetrahydro-1H-inden-5,6-diyl, 1, 2 and/or 3-dimethyl-3a,4,5, 6, 7,7a-hexahydro-1H-inden 5,6-diyls, 1,3-bis(trimethylsilyl)-3a,4,5,6,7,7a-hexahydro-3H-isobenzofuran-5,6-diyl.

Alternatively, the substituents on the said at least one further non adjacent cyclic atom of the non-aromatic bridged hydrocarbyl structure may be a group Y where Y represents a group which is at least as sterically hindering as phenyl and when there are two or more substituents Y they are each as sterically hindering as phenyl and/or combine to form a group which is more sterically hindering than phenyl.

Preferably, Y represents —SR⁴⁰R⁴¹R⁴² wherein S represents Si, C, N, S, O or aryl and R⁴⁰R⁴¹R⁴² are as defined herein. Preferably each Y and/or combination of two or more Y groups is at least as sterically hindering as t-butyl.

More preferably, when there is only one substituent Y, it is at least as sterically hindering as t-butyl whereas where there are two or more substituents Y, they are each at least as sterically hindering as phenyl and at least as sterically hindering as t-butyl if combined into a single group.

Preferably, when S is aryl, R⁴⁰, R⁴¹ and R⁴² are independently hydrogen, alkyl, —BQ³-X³ (X⁴) (wherein B, X³ and X⁴ are as defined herein and Q³ is defined as Q¹ or Q² above), phosphorus, aryl, arylene, alkaryl, arylenalkyl, alkenyl, alkynyl, het, hetero, halo, cyano, nitro, —OR¹⁹, —OC(O)R²⁰, —C(O)R²¹, —C(O)OR²¹, —N(R²³)R²⁴, —C(O)N(R²⁵)R²⁶, —SR²⁹, —C(O)SR³⁰, —C(S)N(R²⁷)R²⁸, —CF₃, —SiR⁷¹R⁷²R⁷³ or alkylphosphorus.

Preferably, when S is Si, C, N, S or R⁴⁰, R⁴¹ and R⁴² are independently hydrogen, alkyl, phosphorus, aryl, arylene, alkaryl, aralkyl, arylenalkyl, alkenyl, alkynyl, het, hetero, halo, cyano, nitro, —OR¹⁹, —OC(O)R²⁰, —C(O)R²¹, —C(O)OR²², —N(R²³)R²⁴, —C(O)N(R²⁵)R²⁶, —SR²⁹, —C(O)SR³⁰, —C(S)N(R²⁷)R²⁸, —CF₃, —SiR⁷¹R⁷²R⁷³, or alkylphosphorus wherein at least one of R⁴-R⁴² is not hydrogen and wherein R¹⁹-R³⁰ are as defined herein; and R⁷¹-R⁷³ are defined as R⁴⁰-R⁴² but are preferably C₁-C₄ alkyl or phenyl.

Preferably, S is Si, C or aryl. However, N, S or O may also be preferred as one or more of the Y groups in combined groups. For the avoidance of doubt, as oxygen or sulphur can be bivalent, R⁴⁰-R⁴² can also be lone pairs.

Preferably, in addition to group Y, the non-aromatic bridged structure may be unsubstituted or further substituted with groups selected from Y, alkyl, aryl, arylene, alkaryl, aralkyl, arylenalkyl, alkenyl, alkynyl, het, hetero, halo, cyano, nitro, —OR¹⁹, —OC(O)R²⁰, —C(O)R²¹, —C(O)OR²², —N(R²³)R²⁴, —C(O)N(R²⁵)R²⁶, —SR²⁹, —C(O)SR³⁰, —C(S)N(R²⁷)R²⁸, —CF₃, —SiR⁷¹R⁷²R⁷³, or alkylphosphorus wherein R¹⁹-R³⁰ are as defined herein; and R⁷¹-R⁷³ are defined as R⁴⁰-R⁴² but are preferably C₁-C₄ alkyl or phenyl.

In addition, when S is aryl, the aryl may be substituted with in addition to R⁴⁰, R⁴¹, R⁴² any of the further substituents defined for the non-aromatic bridged structure above.

More preferred Y substituents may be selected from t-alkyl or t-alkyl, aryl such as -t-butyl, —SiMe₃, or 2-phenylprop-2-yl, -phenyl, alkylphenyl-, phenylalkyl- or phosphinoalkyl- such as phosphinomethyl.

Preferably, when S is Si or C and one or more of R⁴⁰-R⁴² are hydrogen, at least one of R⁴⁰-R⁴² should be sufficiently bulky to give the required steric hindrance and such groups are preferably phosphorus, phosphinoalkyl-, a tertiary carbon bearing group such as -t-butyl, -aryl, -alkaryl, -aralkyl or tertiary silyl.

In some embodiments, there may be two or more said Y substituents on further cyclic atoms of the non-aromatic bridged structure. Optionally, the said two or more substituents may combine to form a further ring structure such as a cycloaliphatic ring structure.

Some typical hydrocarbyl structures are shown below wherein R′, R″, R′″, R″″ etc are defined in the same way as the substituents on the cyclic atoms above but may also be hydrogen, or represent the hetero atom being non substituted if linked directly to a hetero atom and may be the same or different. The diyl methylene linkages to the phosphorus (not shown) are shown in each case.

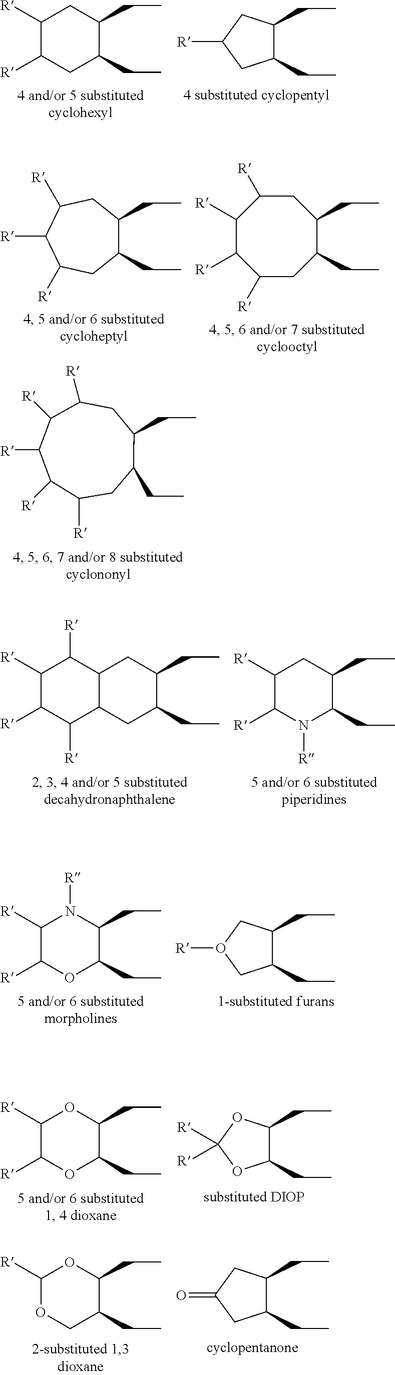

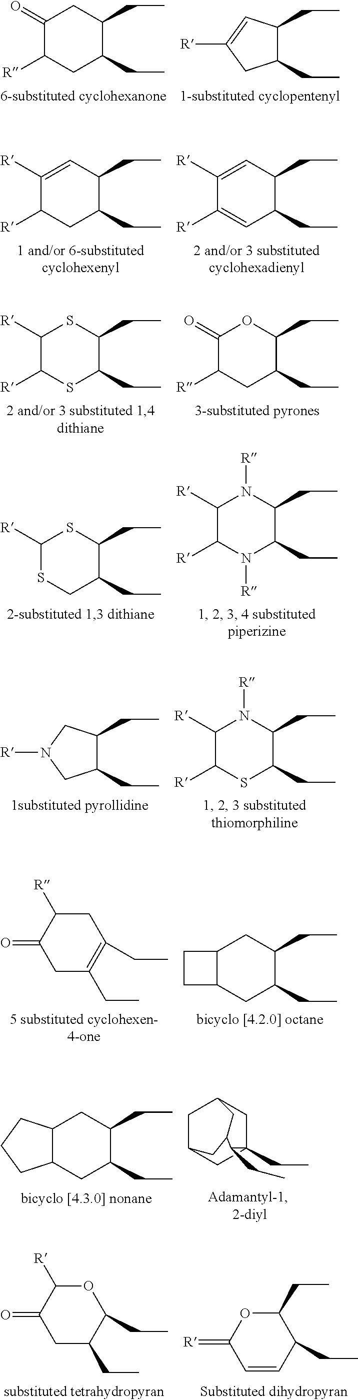

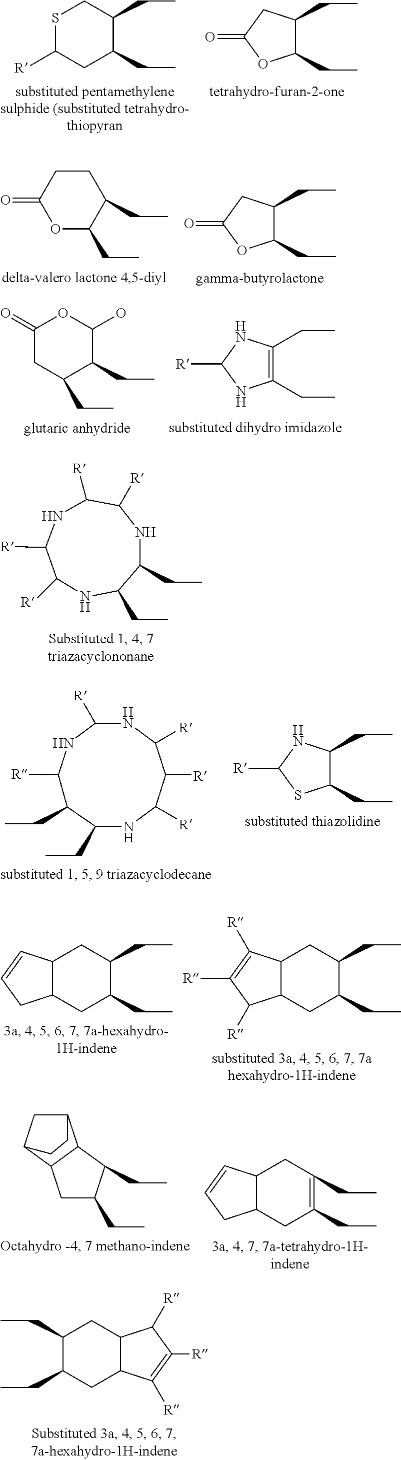

In the structures herein, where there is more than one stereisomeric form possible, all such stereoisomers are intended. However, where there are substituents it is preferable that the at least one substituent on at least one further cyclic atom of the non-aromatic bridged hydrocarbyl structure extends in a trans direction with respect to the A and or B atom ie extends outwardly on the opposite side of the ring.

Preferably, each adjacent cyclic atom to the said available adjacent cyclic atom is not substituted so as to form a further 3-8 atom ring structure via the other adjacent cyclic atom to the said available adjacent cyclic atoms in the at least one ring or via an atom adjacent to the said other adjacent atom but outside the at least one ring in the non-aromatic bridged structure;

An additional preferred set of embodiments is found when R represents an aromatic bridged hydrocarbyl structure ie. having at least one aromatic ring to which Q¹ and Q² are each linked, via the respective linking group, on available adjacent cyclic atoms of the at least one aromatic ring. The aromatic structure may be substituted with one or more substituent(s).

The aromatic bridged hydrocarbyl structure may, where possible, be substituted with one or more substituents selected from alkyl, aryl, Het, halo, cyano, nitro, OR¹⁹, OC(O)R²⁰, C(O)R²¹, C(O)OR²², NR²³R²⁴, C(O)NR²⁵R²⁶, C(S)R²⁵R²⁶, SR²⁷, C(O)SR²⁷, or -J-Q³ (CR¹³(R¹⁴)(R¹⁵)CR¹⁶(R¹⁷)(R¹⁸) where J represents lower alkylene; or two adjacent substituents together with the cyclic atoms of the ring to which they are attached form a further ring, which is optionally substituted by one or more substituents selected from alkyl, halo, cyano, nitro, OR¹⁹, OC(O)R²⁰, C(O)R²¹, C(O)OR²², NR²³R²⁴, C(O)NR²⁵R²⁶, C(S)R²⁵R²⁶, SR²⁷ or C(O)SR²⁷ wherein R¹⁹ to R²⁷ are defined herein.

One type of substituent for the aromatic bridged hydrocarbyl structure is the substituent Y^{x} which may be present on one or more further cyclic atom(s), preferably aromatic cyclic atom of the aromatic bridged cyclic hydrocarbyl structure.

Preferably, when present, the substituent(s) Y^{x} on the aromatic structure has a total ^{X=1-n}ΣtY^{x} of atoms other than hydrogen such that ^{X=1-n}ΣtY^{x} is ≧4, where n is the total number of substituent(s) Y^{x} and tY^{x} represents the total number of atoms other than hydrogen on a particular substituent Y^{x}.

Typically, when there is more than one substituent Y^{x} hereinafter also referred to as simply Y, any two may be located on the same or different cyclic atoms of the aromatic bridged cyclic hydrocarbyl structure. Preferably, there are ≦10 Y groups ie n is 1 to 10, more preferably there are 1-6 Y groups, most preferably 1-4 Y groups on the aromatic structure and, especially, 1, 2 or 3 substituent Y groups on the aromatic structure. The substituted cyclic aromatic atoms may be carbon or hetero but are preferably carbon.

Preferably, when present, ^{X=1-n}ΣtY^{x} is between 4-100, more preferably, 4-60, most preferably, 4-20, especially 4-12.

Preferably, when there is one substituent Y, Y represents a group which is at least as sterically hindering as phenyl and when there are two or more substituents Y they are each as sterically hindering as phenyl and/or combine to form a group which is more sterically hindering than phenyl.

By sterically hindering herein, whether in the context of the groups R¹-R¹² described hereinafter or the substituent Y, or otherwise, we mean the term as readily understood by those skilled in the art but for the avoidance of any doubt, the term more sterically hindering than phenyl can be taken to mean having a lower degree of substitution (DS) than PH₂Ph when PH₂Y (representing the group Y) is reacted with Ni(0) (CO)₄ in eightfold excess according to the conditions below. Similarly, references to more sterically hindering than t-butyl can be taken as references to DS values compared with PH₂t-Bu etc. If, for instance, two Y groups are being compared and PHY¹ is not more sterically hindered than the reference then PHY¹Y² should be compared with the reference. Similarly, if three Y groups are being compared and PHY¹ or PHY¹Y² are not already determined to be more sterically hindered than the standard then PY¹Y²Y³ should be compared. If there are more than three Y groups they should be taken to be more sterically hindered than t-butyl.

Steric hindrance in the context of the invention herein is discussed on page 14 et seq of “Homogenous Transition Metal Catalysis—A Gentle Art”, by C. Masters, published by Chapman and Hall 1981.

Tolman (“Phosphorus Ligand Exchange Equilibria on Zerovalent Nickel. A Dominant Role for Steric Effects”, Journal of American Chemical Society, 92, 1970, 2956-2965) has concluded that the property of the ligands which primarily determines the stability of the Ni(O) complexes is their size rather than their electronic character.

To determine the relative steric hindrance of a group Y or other substituent the method of Tolman to determine DS may be used on the phosphorus analogue of the group to be determined as set out above.

Toluene solutions of Ni(CO)₄ were treated with an eightfold excess of phosphorus ligand; substitution of CO by ligand was followed by means of the carbonyl stretching vibrations in the infrared spectrum. The solutions were equilibriated by heating in sealed tubes for 64 hr at 100°. Further heating at 100° for an additional 74 hrs did not significantly change the spectra. The frequencies and intensities of the carbonyl stretching bands in the spectra of the equilibriated solutions are then determined. The degree of substitution can be estimated semiquantitatively from the relative intensities and the assumption that the extinction coefficients of the bands are all of the same order of magnitude. For example, in the case of P(C₆H₁₁)₃ the A₁ band of Ni(CO)₃L and the B₁ band of Ni(CO)₂L₂ are of about the same intensity, so that the degree of substitution is estimated at 1.5. If this experiment fails to distinguish the respective ligands then the diphenyl phosphorus PPh₂H or di-t-butyl phosphorus should be compared to the PY₂H equivalent as the case may be. Still further, if this also fails to distinguish the ligands then the PPh₃ or P(^{t}Bu)₃ ligand should be compared to PY₃, as the case may be. Such further experimentation may be required with small ligands which fully substitute the Ni(CO)₄ complex.

The group Y may also be defined by reference to its cone angle which can be defined in the context of the invention as the apex angle of a cylindrical cone centred at the midpoint of the aromatic ring. By midpoint is meant a point in the plane of the ring which is equidistant from the cyclic ring atoms.

Preferably, the cone angle of the at least one group Y or the sum of the cone angles of two or more Y groups is at least 10°, more preferably, at least 20°, most preferably, at least 30°. Cone angle should be measured according to the method of Tolman {C. A. Tolman Chem. Rev. 77, (1977), 313-348} except that the apex angle of the cone is now centred at the midpoint of the aromatic ring. This modified use of Tolman cone angles has been used in other systems to measure steric effects such as those in cyclopentadienyl zirconium ethene polymerisation catalysts (Journal of Molecular Catalysis: Chemical 188, (2002), 105-113).

The substituents Y are selected to be of the appropriate size to provide steric hindrance with respect to the active site between the Q¹ and Q² atoms. However, it is not known whether the substituent is preventing the metal leaving, directing its incoming pathway, generally providing a more stable catalytic confirmation, or acting otherwise.

A particularly preferred ligand is found when Y represents —SR⁴⁰R⁴¹R⁴² wherein S represents Si, C, N, S, O or aryl and R⁴⁰R⁴¹R⁴² are as defined hereinafter. Preferably each Y and/or combination of two or more Y groups is at least as sterically hindering as t-butyl.

More preferably, when there is only one substituent Y, it is at least as sterically hindering as t-butyl whereas where there are two or more substituents Y, they are each at least as sterically hindering as phenyl and at least as sterically hindering as t-butyl if considered as a single group.

Preferably, when S is aryl, R⁴⁰, R⁴¹ and R⁴² are independently hydrogen, alkyl, —BQ³-X³ (X⁴) (wherein B, X³ and X⁴ are as defined herein and Q³ is defined as Q¹ or Q² above), phosphorus, aryl, arylene, alkaryl, arylenalkyl, alkenyl, alkynyl, het, hetero, halo, cyano, nitro, —OR¹⁹, —OC(O)R²⁰, —C(O)R²¹, —C(O)OR²², —N(R²³)R²⁴, —C(O)N(R²⁵)R²⁶, —SR²⁹, —C(O)SR³⁰, —C(S)N(R²⁷)R²⁸, —CF₃, —SiR⁷¹R⁷²R⁷³ or alkylphosphorus.

Preferably, when S is Si, C, N, S or O, R⁴⁰, R⁴¹ and R⁴² are independently hydrogen, alkyl, phosphorus, aryl, arylene, alkaryl, aralkyl, arylenalkyl, alkenyl, alkynyl, het, hetero, halo, cyano, nitro, —OR¹⁹, —OC(O)R²⁰, —C(O)R²¹, —C(O)OR²², —N(R²³)R²⁴, —C(O)N(R²⁵)R²⁶, —SR²⁹, —C(O)SR³⁰, —C(S)N(R²⁷)R²⁸, —CF₃, —SiR⁷¹R⁷²R⁷³, or alkylphosphorus wherein at least one of R⁴⁰-R⁴² is not hydrogen and wherein R¹⁹-R³⁰ are as defined herein; and R⁷¹-R⁷³ are defined as R⁴⁰-R⁴² but are preferably C₁-C₄ alkyl or phenyl.

Preferably, S is Si, C or aryl. However, N, S or O may also be preferred as one or more of the Y groups in combined or in the case of multiple Y groups. For the avoidance of doubt, as oxygen or sulphur can be bivalent, R⁴⁰-R⁴² can also be lone pairs.

Preferably, in addition to group Y, the aromatic bridged cyclic hydrocarbyl structure may be unsubstituted or, when possible be further substituted with groups selected from alkyl, aryl, arylene, alkaryl, aralkyl, arylenalkyl, alkenyl, alkynyl, het, hetero, halo, cyano, nitro, —OR¹⁹, —OC(O)R²⁰, —C(O)R²¹, —C(O)OR²², —N(R²³)R²⁴, —C(O)N(R²⁵)R²⁶, —SR²⁹, —C(O)SR³⁰, —C(S)N(R²⁷)R²⁸, —CF₃, —SiR⁷¹R⁷²R⁷³, or alkylphosphorus wherein R¹⁹-R⁴² are as defined herein; and R⁷¹-R⁷³ are defined as R⁴⁰-R⁴² but are preferably C₁-C₄ alkyl or phenyl. In addition, the at least one aromatic ring can be part of a metallocene complex, for instance when R is a cyclopentadienyl or indenyl anion it may form part of a metal complex such as ferrocenyl, ruthenocyl, molybdenocenyl or indenyl equivalents.

Such complexes should be considered as aromatic bridged cyclic hydrocarbyl structures within the context of the present invention and when they include more than one aromatic ring, the substituent(s) Y^{x} or otherwise may be on the same aromatic ring as that to which the Q¹ and Q² atoms are linked or a further aromatic ring of the structure. For instance, in the case of a metallocene, the substituents may be on any one or more rings of the metallocene structure and this may be the same or a different ring than that to which Q¹ and Q² are linked.

Suitable metallocene type ligands which may be substituted as defined herein will be known to the skilled person and are extensively defined in WO 04/024322. A particularly preferred Y substituent for such aromatic anions is when S is Si.

In general, however, when S is aryl, the aryl may be unsubstituted or further substituted with, in addition to R⁴⁰, R⁴¹, R⁴², any of the further substituents defined for the aromatic structure above.

More preferred Y substituents in the present invention may be selected from t-alkyl or t-alkyl, aryl such as -t-butyl or 2-phenylprop-2-yl, —SiMe₃, -phenyl, alkylphenyl-, phenylalkyl- or phosphinoalkyl- such as phosphinomethyl.

Preferably, when S is Si or C and one or more of R⁴⁰-R⁴² are hydrogen, at least one of R⁴⁰-R⁴² should be sufficiently bulky to give the required steric hindrance and such groups are preferably phosphorus, phosphinoalkyl-, a tertiary carbon bearing group such as -t-butyl, -aryl, -alkaryl, -aralkyl or tertiary silyl.

Preferably, the aromatic bridged cyclic hydrocarbyl structure has, including substituents, from 5 up to 70 cyclic atoms, more preferably, 5 to 40 cyclic atoms, most preferably, 5-22 cyclic atoms; especially 5 or 6 cyclic atoms, if not a metallocene complex.

Preferably, the aromatic bridged cyclic hydrocarbyl structure may be monocyclic or polycyclic. The cyclic aromatic atoms may be carbon or hetero, wherein references to hetero herein are references to sulphur, oxygen and/or nitrogen. However, it is preferred that the Q¹ and Q² atoms are linked to available adjacent cyclic carbon atoms of the at least one aromatic ring. Typically, when the cyclic hydrocarbyl structure is polycylic it is preferably bicyclic or tricyclic. The further cycles in the aromatic bridged cyclic hydrocarbyl structure may or may not themselves be aromatic and the term aromatic bridged cyclic hydrocarbyl structure should be understood accordingly. A non-aromatic cyclic ring(s) as defined herein may include unsaturated bonds. By cyclic atom is meant an atom which forms part of a cyclic skeleton.

Preferably, the aromatic bridged cyclic hydrocarbyl structure whether substituted or otherwise preferably comprises less than 200 atoms, more preferably, less than 150 atoms, more preferably, less than 100 atoms.

By the term one further cyclic atom of the aromatic bridged hydrocarbyl structure is meant any further cyclic atom in the aromatic structure which is not an available adjacent cyclic atom of the at least one aromatic ring to which the Q¹ or Q² atoms are linked, via the linking group.

As mentioned above, the immediate adjacent cyclic atoms on either side of the said available adjacent cyclic atoms are preferably not substituted. As an example, an aromatic phenyl ring joined to a Q¹ atom via position 1 on the ring and joined to a Q² atom via position 2 on the ring has preferably one or more said further aromatic cyclic atoms substituted at ring position 4 and/or 5 and two immediate adjacent cyclic atoms to the said available adjacent cyclic atoms not substituted at positions 3 and 6. However, this is only a preferred substituent arrangement and substitution at ring positions 3 and 6, for example, is possible.

The term aromatic ring or aromatic bridged means that the at least one ring or bridge to which the Q¹ and Q² atom are immediately linked via B & A respectively is aromatic, and aromatic should preferably be interpreted broadly to include not only a phenyl, cyclopentadienyl anion, pyrollyl, pyridinyl, type structures but other rings with aromaticity such as that found in any ring with delocalised Pi electrons able to move freely in the said ring.

Preferred aromatic rings have 5 or 6 atoms in the ring but rings with 4n+2 pi electrons are also possible such as [1,4] annulene, [1,8] annulene, etc

The aromatic bridged cyclic hydrocarbyl structure may be selected from benzene-1,2 diyl, ferrocene-1,2-diyl, naphthalene-1,2-diyl, 4 or 5 methyl benzene-1,2-diyl, 1′-methyl ferrocene-1,2-diyl, 4 and/or 5 t-alkylbenzene-1,2-diyl, 4,5-diphenyl-benzene-1,2-diyl, 4 and/or 5-phenyl-benzene-1,2-diyl, 4,5-di-t-butyl-benzene-1,2-diyl, 4 or 5-t-butylbenzene-1,2-diyl, 2, 3, 4 and/or 5 t-alkyl-naphthalene-8,9-diyl, 1H-inden-5,6-diyl, 1, 2 and/or 3 methyl-1H-inden-5,6-diyl, 4,7 methano-1H-indene-1,2-diyl, 1, 2 and/or 3-dimethyl-1H-inden 5,6-diyls, 1,3-bis(trimethylsilyl)-isobenzofuran-5,6-diyl, 4-(trimethylsilyl)benzene-1,2 diyl, 4-phosphinomethyl benzene-1,2 diyl, 4-(2′-phenylprop-2′-yl)benzene-1,2 diyl, 4-dimethylsilylbenzene-1,2diyl, 4-di-t-butyl, methylsilyl benzene-1,2diyl, 4-(t-butyldimethylsilyl)-benzene-1,2diyl, 4-t-butylsilyl-benzene-1,2diyl, 4-(tri-t-butylsilyl)-benzene-1,2diyl, 4-(2′-tert-butylprop-2′-yl)benzene-1,2 diyl, 4-(2′,2′,3′, 4′,4′ pentamethyl-pent-3′-yl)-benzene-1,2diyl, 4-(2′,2′,4′,4′-tetramethyl,3′-t-butyl-pent-3′-yl)-benzene-1,2 diyl, 4-(or 1′)ₜ-alkylferrocene-1,2-diyl, 4,5-diphenyl-ferrocene-1,2-diyl, 4-(or 1′)phenyl-ferrocene-1,2-diyl, 4,5-di-t-butyl-ferrocene-1,2-diyl, 4-(or 1′)ₜ-butylferrocene-1,2-diyl, 4-(or 1′)(trimethylsilyl) ferrocene-1,2 diyl, 4-(or 1′)phosphinomethyl ferrocene-1,2 diyl, 4-(or 1′)(2′-phenylprop-2′-yl) ferrocene-1,2 diyl, 4-(or 1′)dimethylsilylferrocene-1,2diyl, 4-(or 1′)di-t-butyl, methylsilyl ferrocene-1,2diyl, 4-(or 1′)(t-butyldimethylsilyl)-ferrocene-1,2diyl, 4-(or 1′)ₜ-butylsilyl-ferrocene-1,2diyl, 4-(or 1′)(tri-t-butylsilyl)-ferrocene-1,2diyl, 4-(or 1′)(2′-tert-butylprop-2′-yl)ferrocene-1,2 diyl, 4-(or 1′) (2′,2′,3′,4′, 4′ pentamethyl-pent-3′-yl)-ferrocene-1,2diyl, 4-(or 1′)(2′,2′,4′,4′-tetramethyl,3′-t-butyl-pent-3′-yl)-ferrocene-1,2 diyl.

In the structures herein, where there is more than one stereisomeric form possible, all such stereoisomers are intended.

As mentioned above, in some embodiments, there may be two substituents on further cyclic atoms of the aromatic structure. Optionally, the said two or more substituents may, especially when on neighbouring cyclic atoms, combine to form a further ring structure such as a cycloaliphatic ring structure.

Such cycloaliphatic ring structures may be saturated or unsaturated, bridged or unbridged, substituted with alkyl, Y groups as defined herein, aryl, arylene, alkaryl, aralkyl, arylenalkyl, alkenyl, alkynyl, het, hetero, halo, cyano, nitro, —OR¹⁹, —OC(O)R²⁰, —C(O)R²¹, —C(O)OR²², —N(R²³)R²⁴, —C(O)N(R²⁵)R²⁶, —SR²⁹, —C(O)SR³⁰, —C(S)N(R²⁷)R²⁸, —CF₃, —SiR⁷¹R⁷²R⁷³, or phosphinoalkyl wherein, when present, at least one of R⁴⁰-R⁴² is not hydrogen and wherein R¹⁹-R³⁰ are as defined herein; and R⁷¹-R⁷³ are defined as R⁴⁰-R⁴² but are preferably C₁-C₄ alkyl or phenyl and/or be interrupted by one or more (preferably less than a total of 4) oxygen, nitrogen, sulphur, silicon atoms or by silano or dialkyl silicon groups or mixtures thereof.

Examples of such structures include piperidine, pyridine, morpholine, cyclohexane, cycloheptane, cyclooctane, cyclononane, furan, dioxane, alkyl substituted DIOP, 2-alkyl substituted 1,3 dioxane, cyclopentanone, cyclohexanone, cyclopentene, cyclohexene, cyclohexadiene, 1,4 dithiane, piperizine, pyrollidine, thiomorpholine, cyclohexenone, bicyclo[4.2.0]octane, bicyclo[4.3.0]nonane, adamantane, tetrahydropyran, dihydropyran, tetrahydrothiopyran, tetrahydro-furan-2-one, delta valerolactone, gamma-butyrolactone, glutaric anhydride, dihydroimidazole, triazacyclononane, triazacyclodecane, thiazolidine, hexahydro-1H-indene (5,6 diyl), octahydro-4,7 methano-indene (1,2 diyl) and tetrahydro-1H-indene (5,6 diyl) all of which may be unsubstituted or substituted as defined for aryl herein.

Specific but non-limiting examples of unsubstituted aromatic bridged bidentate ligands within this invention include the following: 1,2-bis-(di-tert-butylphosphinomethyl)benzene, 1,2-bis-(di-tert-pentylphosphinomethyl)benzene, 1,2-bis-(di-tert-butylphosphinomethyl)naphthalene, 1,2 bis(diadamantylphosphinomethyl)benzene, 1,2 bis(di-3,5-dimethyladamantylphosphinomethyl)benzene, 1,2 bis(di-5-tert-butyladamantylphosphinomethyl)benzene, 1,2 bis(1-adamantyl tert-butyl-phosphinomethyl)benzene, 1-(diadamantylphosphinomethyl)-2-(di-tert-butylphosphinomethyl)benzene, 1-(di-tert-butylphosphinomethyl)-2-(dicongressylphosphinomethyl)benzene, 1-(di-tert-butylphosphino)-2-(phospha-adamantyl)o-xylene, 1-(diadamantylphosphino)-2-(phospha-adamantyl)o-xylene, 1-(di-tert-butylphosphinomethyl)-2-(phospha-adamantyl)benzene, 1-(diadamantylphosphinomethyl)-2-(phospha-adamantyl)benzene, 1-(phospha-adamantyl)-2-(phospha-adamantyl)methylbenzene, 1-(di-tert-butylphosphinomethyl)-2-(di-tert-butylphosphino)benzene, 1-(diadamantylphosphinomethyl)-2-(diadamantylphosphino)benzene, 1-(di-tert-butylphosphinomethyl)-2-(diadamantylphosphino)benzene, 1-(di-tert-butylphosphino)-2-(P-(2,2,6,6-tetramethyl-phospha-cyclohexan-4-one) o-xylene, 1-(di-tert-butylphosphinomethyl)-2-(P-(2,2,6,6-tetramethyl-phospha-cyclohexan-4-one) benzene, 1-(2-(P-(2,2,6,6-tetramethyl-phospha-cyclohexan-4-one))-benzyl)-2,2,6,6-tetramethyl-phospha-cyclohexan-4-one, 1-(tert-butyl,adamantylphosphinomethyl)-2-(di-adamantylphosphinomethyl)benzene and 1-(P-(2,2,6,6-tetramethyl-phospha-cyclohexan-4-one)-2-(phospha-adamantyl)o-xylyl—wherein “phospha-adamantyl” is selected from 2-phospha-1,3,5,7-tetramethyl-6,9,10-trioxadamantyl,2-phospha-1,3,5-trimethyl-6,9,10 trioxadamantyl, 2-phospha-1,3,5,7-tetra(trifluoromethyl)-6,9,10-trioxadamantyl or 2-phospha-1,3,5-tri(trifluoromethyl)-6,9,10-trioxadamantyl-, 1,2-bis-(ditertbutylphosphinomethyl)ferrocene, 1,2,3-tris-(ditertbutylphosphinomethyl)ferrocene, 1,2-bis(1,3,5,7-tetramethyl-6,9,10-trioxa-2-phospha-adamantylmethyl)ferrocene, 1,2-bis-α,α-(P-(2,2,6,6-tetramethyl-phospha-cyclohexan-4-one))dimethylferrocene and 1-(ditertbutylphosphinomethyl)-2-(P-(2,2,6,6-tetramethyl-phospha-cyclohexan-4-one))ferrocene and 1,2-bis(1,3,5,7-tetramethyl-6,9,10-trioxa-2-phospha-adamantylmethyl)benzene.

Examples of suitable substituted non-aromatic bridged bidentate ligands are cis-1,2-bis(di-t-butylphosphinomethyl)-4,5-dimethyl cyclohexane; cis-1,2-bis(di-t-butylphosphinomethyl)-5-methylcyclopentane; cis-1,2-bis(2-phosphinomethyl-1,3,5,7-tetramethyl-6,9,10-trioxa-adamantyl)-4,5-dimethylcyclohexane; cis-1,2-bis(2-phosphinomethyl-1,3,5,7-tetramethyl-6,9,10-trioxa-adamantyl) 5-methylcyclopentane; cis-1,2-bis(di-adamantylphosphinomethyl)-4,5 dimethylcyclohexane; cis-1,2-bis(di-adamantylphosphinomethyl)-5-methyl cyclopentane; cis-1-(P,P adamantyl, t-butyl phosphinomethyl)-2-(di-t-butylphosphinomethyl)-4,5-dimethylcyclohexane; cis-1-(P,P adamantyl, t-butyl phosphinomethyl)-2-(di-t-butylphosphinomethyl)-5-methylcyclopentane; cis-1-(2-phosphinomethyl-1,3,5,7-tetramethyl-6,9,10-trioxa-adamantyl)-2-(di-t-butylphosphinomethyl)-4,5-dimethylcyclohexane; cis-1-(2-phosphinomethyl-1,3,5,7-tetramethyl-6,9,10-trioxa-adamantyl)-2-(di-t-butylphosphinomethyl)-5-methyl cyclopentane; cis-1-(2-phosphinomethyl-1,3,5,7-tetramethyl-6,9,10-trioxa-adamantyl)-2-(diadamantylphosphinomethyl)-5-methyl cyclohexane; cis-1-(2-phosphinomethyl-1,3,5,7-tetramethyl-6,9,10-trioxa-adamantyl)-2-(diadamantylphosphinomethyl)-5-methyl cyclopentane; cis-1-(2-phosphinomethyl-1,3,5,7-tetramethyl-6,9,10-trioxa-adamantyl)-2-(diadamantylphosphinomethyl)cyclobutane; cis-1-(di-t-butylphosphinomethyl)-2-(diadamantylphosphinomethyl)-4,5-dimethyl cyclohexane; cis-1-(di-t-butylphosphinomethyl)-2-(diadamantylphosphinomethyl)-5-methyl cyclopentane; cis-1,2-bis(2-phosphinomethyl-1,3,5-trimethyl-6,9,10-trioxatricyclo-{3.3.1.1[3.7]}decyl)-4,5-dimethyl cyclohexane; cis-1,2-bis(2-phosphinomethyl-1,3,5-trimethyl-6,9,10-trioxatricyclo-{3.3.1.1[3.7]}decyl)-5-methyl cyclopentane; cis-1-(2-phosphinomethyl-1,3,5-trimethyl-6,9,10-trioxatricyclo-{3.3.1.1[3.7]}decyl)-2-(di-t-butylphosphinomethyl)-4,5-dimethyl cyclohexane; cis-1-(2-phosphinomethyl-1,3,5-trimethyl-6,9,10-trioxatricyclo-{3.3.1.1[3.7]}decyl)-2-(di-t-butylphosphinomethyl)-5-methyl cyclopentane; cis-1-(2-phosphinomethyl-1,3,5-trimethyl-6,9,10-trioxatricyclo-{3.3.1.1[3.7]}decyl)-2-(diadamantylphosphinomethyl)-4,5-dimethyl cyclohexane; cis-1-(2-phosphinomethyl-1,3,5-trimethyl-6,9,10-trioxatricyclo-{3.3.1.1[3.7]}decyl)-2-(diadamantylphosphinomethyl)-5-methyl cyclopentane; cis-1,2-bis-perfluoro(2-phosphinomethyl-1,3,5,7-tetramethyl-6,9,10-trioxatricyclo{3.3.1.1[3.7]}-decyl)-4,5-dimethyl cyclohexane; cis-1,2-bis-perfluoro(2-phosphinomethyl-1,3,5,7-tetramethyl-6,9,10-trioxatricyclo{3.3.1.1[3.7]}decyl)-5-methyl cyclopentane; cis-1,2-bis-(2-phosphinomethyl-1,3,5,7-tetra(trifluoro-methyl)-6,9,10-trioxatricyclo{3.3.1.1[3.7]}decyl)-4,5-dimethyl cyclohexane; cis-1,2-bis-(2-phosphinomethyl-1,3,5,7-tetra(trifluoro-methyl)-6,9,10-trioxatricyclo{3.3.1.1[3.7]}decyl)-5-methyl cyclopentane; cis-1-(2-phosphino-1,3,5,7-tetramethyl-6,9,10-trioxa-adamantyl)-2-(di-t-butylphosphinomethyl)-4,5-dimethylcyclohexane; cis-1-(2-phosphinomethyl-1,3,5,7-tetramethyl-6,9,10-trioxa-adamantyl)-2-(2-phosphino-1,3,5,7-tetramethyl-6,9,10-trioxa-adamantyl)-4,5-dimethyl cyclohexane; cis-1-(di-t-butylphosphino)-2-(di-t-butylphosphinomethyl)-4,5-dimethyl cyclohexane; cis-1-(di-adamantylphosphino)-2-(di-t-butylphosphinomethyl)-4,5-dimethyl cyclohexane; cis-1-(di-adamantylphosphino)-2-(di-adamantylphosphinomethyl)-4,5-dimethyl cyclohexane; cis-1-(2-phosphino-1,3,5,7-tetramethyl-6,9,10-trioxa-adamantyl)-2-(di-adamantylphosphinomethyl)-4,5-dimethyl cyclohexane; cis-1-(P-(2,2,6,6-tetramethyl-phospha-cyclohexan-4-one))-2-(di-t-butylphosphinomethyl) 4,5-dimethyl cyclohexane; 1-[4,5-dimethyl-2-P-(2,2,6,6-tetramethyl-phospha-cyclohexan-4-one)-[1S,2R]cyclohexylmethyl]-P-2,2,6,6-tetramethyl-phospha-cyclohexan-4-one.

Examples of suitable non-substituted non-aromatic bridged bidentate ligands are cis-1,2-bis(di-t-butylphosphinomethyl)cyclohexane; cis-1,2-bis(di-t-butylphosphinomethyl)cyclopentane; cis-1,2-bis(di-t-butylphosphinomethyl)cyclobutane; cis-1,2-bis(2-phosphinomethyl-1,3,5,7-tetramethyl-6,9,10-trioxa-adamantyl)cyclohexane; cis-1,2-bis(2-phosphinomethyl-1,3,5,7-tetramethyl-6,9,10-trioxa-adamantyl)cyclopentane; cis-1,2-bis(2-phosphinomethyl-1,3,5,7-tetramethyl-6,9,10-trioxa-adamantyl)cyclobutane; cis-1,2-bis(di-adamantylphosphinomethyl)cyclohexane; cis-1,2-bis(di-adamantylphosphinomethyl)cyclopentane; cis-1,2-bis(di-adamantylphosphinomethyl)cyclobutane; cis-1-(P,P-adamantyl, t-butyl-phosphinomethyl)-2-(di-t-butylphosphinomethyl)cyclohexane; cis-1-(2-phosphino-1,3,5,7-tetramethyl-6,9,10-trioxa-adamantyl)-2-(di-t-butylphosphinomethyl)cyclohexane; cis-1-(2-phosphinomethyl-1,3,5,7-tetramethyl-6,9,10-trioxa-adamantyl)-2-(2-phosphino-1,3,5,7-tetramethyl-6,9,10-trioxa-adamantyl)cyclohexane; cis-1-(di-t-butylphosphino)-2-(di-t-butylphosphinomethyl)cyclohexane; cis-1-(di-adamantylphosphino)-2-(di-t-butylphosphinomethyl)cyclohexane; cis-1-(di-adamantylphosphino)-2-(di-adamantylphosphinomethyl)cyclohexane; cis-1-(2-phosphino-1,3,5,7-tetramethyl-6,9,10-trioxa-adamantyl)-2-(di-adamantylphosphinomethyl)cyclohexane; cis-1-(P-(2,2,6,6-tetramethyl-phospha-cyclohexan-4-one))-2-(di-t-butylphosphinomethyl)cyclohexane; cis-1-(P-(2,2,6,6-tetramethyl-phospha-cyclohexan-4-one))-2-(P-(2,2,6,6-tetramethyl-phospha-cyclohexan-4-one))methylcyclohexane; cis-1-(P,P-adamantyl, t-butyl-phosphinomethyl)-2-(di-t-butylphosphinomethyl)cyclopentane; cis-1-(P,P-adamantyl, t-butyl-phosphinomethyl)-2-(di-t-butylphosphinomethyl)cyclobutane; cis-1-(2-phosphinomethyl-1,3,5,7-tetramethyl-6,9,10-trioxa-adamantyl)-2-(di-t-butylphosphinomethyl)cyclohexane; cis-1-(2-phosphinomethyl-1,3,5,7-tetramethyl-6,9,10-trioxa-adamantyl)-2-(di-t-butylphosphinomethyl)cyclopentane; cis-1-(2-phosphinomethyl-1,3,5,7-tetramethyl-6,9,10-trioxa-adamantyl)-2-(di-t-butylphosphinomethyl)cyclobutane; cis-1-(2-phosphinomethyl-1,3,5,7-tetramethyl-6,9,10-trioxa-adamantyl)-2-(diadamantylphosphinomethyl)cyclohexane; cis-1-(2-phosphinomethyl-1,3,5,7-tetramethyl-6,9,10-trioxa-adamantyl)-2-(diadamantylphosphinomethyl)cyclopentane; cis-1-(2-phosphinomethyl-1,3,5,7-tetramethyl-6,9,10-trioxa-adamantyl)-2-(diadamantylphosphinomethyl)cyclobutane; cis-1-(di-t-butylphosphinomethyl)-2-(diadamantylphosphinomethyl)cyclohexane; cis-1-(di-t-butylphosphinomethyl)-2-(diadamantylphosphinomethyl)cyclopentane; cis-1-(di-t-butylphosphinomethyl)-2-(diadamantylphosphinomethyl)cyclobutane; cis-1,2-bis(2-phosphinomethyl-1,3,5-trimethyl-6,9,10-trioxatricyclo-{3.3.1.1[3.7]}decyl)cyclohexane; cis-1,2-bis(2-phosphinomethyl-1,3,5-trimethyl-6,9,10-trioxatricyclo-{3.3.1.1[3.7]}decyl)cyclopentane; cis-1,2-bis(2-phosphinomethyl-1,3,5-trimethyl-6,9,10-trioxatricyclo-{3.3.1.1[3.7]}decyl)cyclobutane; cis-1-(2-phosphinomethyl-1,3,5-trimethyl-6,9,10-trioxatricyclo-{3.3.1.1[3.7]}decyl)-2-(di-t-butylphosphinomethyl)cyclohexane; cis-1-(2-phosphinomethyl-1,3,5-trimethyl-6,9,10-trioxatricyclo-{3.3.1.1[3.7]}decyl)-2-(di-t-butylphosphinomethyl)cyclopentane; cis-1-(2-phosphinomethyl-1,3,5-trimethyl-6,9,10-trioxatricyclo-{3.3.1.1[3.7]}decyl)-2-(di-t-butylphosphinomethyl)cyclobutane; cis-1-(2-phosphinomethyl-1,3,5-trimethyl-6,9,10-trioxatricyclo-{3.3.1.1[3.7]}decyl)-2-(diadamantylphosphinomethyl)cyclohexane; cis-1-(2-phosphinomethyl-1,3,5-trimethyl-6,9,10-trioxatricyclo-{3.3.1.1[3.7]}decyl)-2-(diadamantylphosphinomethyl)cyclopentane; cis-1-(2-phosphinomethyl-1,3,5-trimethyl-6,9,10-trioxatricyclo-{3.3.1.1[3.7]}decyl)-2-(diadamantylphosphinomethyl)cyclobutane; cis-1,2-bis-perfluoro(2-phosphinomethyl-1,3,5,7-tetramethyl-6,9,10-trioxatricyclo{3.3.1.1[3.7]}-decyl)cyclohexane; cis-1,2-bis-perfluoro(2-phosphinomethyl-1,3,5,7-tetramethyl-6,9,10-trioxatricyclo{3.3.1.1[3.7]}decyl)cyclopentane; cis-1,2-bis-perfluoro(2-phosphinomethyl-1,3,5,7-tetramethyl-6,9,10-trioxatricyclo{3.3.1.1[3.7]}decyl)cyclobutane; cis-1,2-bis-(2-phosphinomethyl-1,3,5,7-tetra(trifluoro-methyl)-6,9,10-trioxatricyclo{3.3.1.1[3.7]}decyl)cyclohexane; cis-1,2-bis-(2-phosphinomethyl-1,3,5,7-tetra(trifluoro-methyl)-6,9,10-trioxatricyclo{3.3.1.1[3.7]}decyl)cyclopentane; and cis-1,2-bis-(2-phosphinomethyl-1,3,5,7-tetra(trifluoro-methyl)-6,9,10-trioxatricyclo{3.3.1.1[3.7]}decyl)cyclobutane, (2-exo, 3-exo)-bicyclo[2.2.1]heptane-2,3-bis(di-tert-butylphosphinomethyl) and (2-endo, 3-endo)-bicyclo[2.2.1]heptane-2,3-bis(di-tert-butylphosphinomethyl).

Examples of substituted aromatic bridged ligands in accordance with the invention include 1,2-bis(di-t-butylphosphinomethyl)-4,5-diphenyl benzene; 1,2-bis(di-t-butylphosphinomethyl)-4-phenylbenzene; 1,2-bis(di-t-butylphosphinomethyl)-4,5-bis-(trimethylsilyl)benzene; 1,2-bis(di-t-butylphosphinomethyl)-4-(trimethylsilyl)benzene; 1,2-bis(2-phosphinomethyl-1,3,5,7-tetramethyl-6,9,10-trioxa-adamantyl)-4,5-diphenylbenzene; 1,2-bis(2-phosphinomethyl-1,3,5,7-tetramethyl-6,9,10-trioxa-adamantyl)-4-phenylbenzene; 1,2-bis(2-phosphinomethyl-1,3,5,7-tetramethyl-6,9,10-trioxa-adamantyl)-4,5-bis-(trimethylsilyl)benzene; 1,2-bis(2-phosphinomethyl-1,3,5,7-tetramethyl-6,9,10-trioxa-adamantyl)-4-(trimethylsilyl)benzene; 1,2-bis(di-adamantylphosphinomethyl)-4,5 diphenylbenzene; 1,2-bis(di-adamantylphosphinomethyl)-4-phenyl benzene; 1,2-bis(di-adamantylphosphinomethyl)-4,5 bis-(trimethylsilyl)benzene; 1,2-bis(di-adamantylphosphinomethyl)-4-(trimethylsilyl)benzene; 1-(P,P adamantyl, t-butyl phosphinomethyl)-2-(di-t-butylphosphinomethyl)-4,5-diphenylbenzene; 1-(P,P adamantyl, t-butyl phosphinomethyl)-2-(di-t-butylphosphinomethyl)-4-phenylbenzene; 1-(P,P adamantyl, t-butyl phosphinomethyl)-2-(di-t-butylphosphinomethyl)-4,5-bis-(trimethylsilyl)benzene; 1-(P,P adamantyl, t-butyl phosphinomethyl)-2-(di-t-butylphosphinomethyl)-4-(trimethylsilyl)benzene; 1-(2-phosphinomethyl-1,3,5,7-tetramethyl-6,9,10-trioxa-adamantyl)-2-(di-t-butylphosphinomethyl)-4,5-diphenylbenzene; 1-(2-phosphinomethyl-1,3,5,7-tetramethyl-6,9,10-trioxa-adamantyl)-2-(di-t-butylphosphinomethyl)-4-phenyl benzene; 1-(2-phosphinomethyl-1,3,5,7-tetramethyl-6,9,10-trioxa-adamantyl)-2-(di-t-butylphosphinomethyl)-4,5-bis-(trimethylsilyl)benzene; 1-(2-phosphinomethyl-1,3,5,7-tetramethyl-6,9,10-trioxa-adamantyl)-2-(di-t-butylphosphinomethyl)-4-(trimethylsilyl)benzene; 1-(2-phosphinomethyl-1,3,5,7-tetramethyl-6,9,10-trioxa-adamantyl)-2-(diadamantylphosphinomethyl)-4,5-diphenyl benzene; 1-(2-phosphinomethyl-1,3,5,7-tetramethyl-6,9,10-trioxa-adamantyl)-2-(diadamantylphosphinomethyl)-4-phenyl benzene; 1-(2-phosphinomethyl-1,3,5,7-tetramethyl-6,9,10-trioxa-adamantyl)-2-(diadamantylphosphinomethyl)-4,5-bis-(trimethylsilyl)benzene; 1-(2-phosphinomethyl-1,3,5,7-tetramethyl-6,9,10-trioxa-adamantyl)-2-(diadamantylphosphinomethyl)-4-(trimethylsilyl)benzene; 1-(di-t-butylphosphinomethyl)-2-(diadamantylphosphinomethyl)-4,5-diphenyl benzene; 1-(di-t-butylphosphinomethyl)-2-(diadamantylphosphinomethyl)-4-phenyl benzene; 1-(di-t-butylphosphinomethyl)-2-(diadamantylphosphinomethyl)-4,5-bis-(trimethylsilyl)benzene; 1-(di-t-butylphosphinomethyl)-2-(diadamantylphosphinomethyl)-4-(trimethylsilyl)benzene; 1,2-bis(2-phosphinomethyl-1,3,5-trimethyl-6,9,10-trioxatricyclo-{3.3.1.1[3.7]}decyl)-4,5-diphenyl benzene; 1,2-bis(2-phosphinomethyl-1,3,5-trimethyl-6,9,10-trioxatricyclo-{3.3.1.1[3.7]}decyl)-4-phenyl benzene; 1,2-bis(2-phosphinomethyl-1,3,5-trimethyl-6,9,10-trioxatricyclo-{3.3.1.1[3.7]}decyl)-4,5-bis-(trimethylsilyl)benzene; 1,2-bis(2-phosphinomethyl-1,3,5-trimethyl-6,9,10-trioxatricyclo-{3.3.1.1[3.7]}decyl)-4-(trimethylsilyl)benzene; 1-(2-phosphinomethyl-1,3,5-trimethyl-6,9,10-trioxatricyclo-{3.3.1.1[3.7]}decyl)-2-(di-t-butylphosphinomethyl)-4,5-diphenyl benzene; 1-(2-phosphinomethyl-1,3,5-trimethyl-6,9,10-trioxatricyclo-{3.3.1.1[3.7]}decyl)-2-(di-t-butylphosphinomethyl)-4-phenyl benzene; 1-(2-phosphinomethyl-1,3,5-trimethyl-6,9,10-trioxatricyclo-{3.3.1.1[3.7]}decyl)-2-(di-t-butylphosphinomethyl)-4,5-bis-(trimethylsilyl)benzene; 1-(2-phosphinomethyl-1,3,5-trimethyl-6,9,10-trioxatricyclo-{3.3.1.1[3.7]}decyl)-2-(di-t-butylphosphinomethyl)-4-(trimethylsilyl)benzene; 1-(2-phosphinomethyl-1,3,5-trimethyl-6,9,10-trioxatricyclo-{3.3.1.1[3.7]}decyl)-2-(diadamantylphosphinomethyl)-4,5-diphenyl benzene; 1-(2-phosphinomethyl-1,3,5-trimethyl-6,9,10-trioxatricyclo-{3.3.1.1[3.7]}decyl)-2-(diadamantylphosphinomethyl)-4-phenyl benzene; 1-(2-phosphinomethyl-1,3,5-trimethyl-6,9,10-trioxatricyclo-{3.3.1.1[3.7]}decyl)-2-(diadamantylphosphinomethyl)-4,5-bis-(trimethylsilyl)benzene; 1-(2-phosphinomethyl-1,3,5-trimethyl-6,9,10-trioxatricyclo-{3.3.1.1[3.7]}decyl)-2-(diadamantylphosphinomethyl)-4-(trimethylsilyl)benzene; 1,2-bis-perfluoro(2-phosphinomethyl-1,3,5,7-tetramethyl-6,9,10-trioxatricyclo{3.3.1.1[3.7]}-decyl)-4,5-diphenyl benzene; 1,2-bis-perfluoro(2-phosphinomethyl-1,3,5,7-tetramethyl-6,9,10-trioxatricyclo{3.3.1.1[3.7]}decyl)-4-phenyl benzene; 1,2-bis-perfluoro(2-phosphinomethyl-1,3,5,7-tetramethyl-6,9,10-trioxatricyclo{3.3.1.1[3.7]}-decyl)-4,5-bis-(trimethylsilyl)benzene; 1,2-bis-perfluoro(2-phosphinomethyl-1,3,5,7-tetramethyl-6,9,10-trioxatricyclo{3.3.1.1[3.7]}decyl)-4-(trimethylsilyl)benzene; 1,2-bis-(2-phosphinomethyl-1,3,5,7-tetra(trifluoro-methyl)-6,9,10-trioxatricyclo{3.3.1.1[3.7]}decyl)-4,5-diphenyl benzene; 1,2-bis-(2-phosphinomethyl-1,3,5,7-tetra(trifluoro-methyl)-6,9,10-trioxatricyclo{3.3.1.1[3.7]}decyl)-4-phenyl benzene; 1,2-bis-(2-phosphinomethyl-1,3,5,7-tetra(trifluoro-methyl)-6,9,10-trioxatricyclo{3.3.1.1[3.7]}decyl)-4,5-bis-(trimethylsilyl)benzene; 1,2-bis-(2-phosphinomethyl-1,3,5,7-tetra(trifluoro-methyl)-6,9,10-trioxatricyclo{3.3.1.1[3.7]}decyl)-4-(trimethylsilyl)benzene; 1,2-bis(di-t-butylphosphinomethyl)-4,5-di-(2′-phenylprop-2′-yl)benzene; 1,2-bis(di-t-butylphosphinomethyl)-4-(2′-phenylprop-2′-yl)benzene; 1,2-bis(di-t-butylphosphinomethyl)-4,5-di-t-butyl benzene; 1,2-bis(di-t-butylphosphinomethyl)-4-t-butylbenzene; 1,2-bis(2-phosphinomethyl-1,3,5,7-tetramethyl-6,9,10-trioxa-adamantyl)-4,5-di-(2′-phenylprop-2′-yl)benzene; 1,2-bis(2-phosphinomethyl-1,3,5,7-tetramethyl-6,9,10-trioxa-adamantyl)-4-(2′-phenylprop-2′-yl)benzene; 1,2-bis(2-phosphinomethyl-1,3,5,7-tetramethyl-6,9,10-trioxa-adamantyl)-4,5-(di-t-butyl)benzene; 1,2-bis(2-phosphinomethyl-1,3,5,7-tetramethyl-6,9,10-trioxa-adamantyl)-4-t-butylbenzene; 1,2-bis(di-adamantylphosphinomethyl)-4,5-di-(2′-phenylprop-2′-yl)benzene; 1,2-bis(di-adamantylphosphinomethyl)-4-(2′-phenylprop-2′-yl)benzene; 1,2-bis(di-adamantylphosphinomethyl)-4,5-(di-t-butyl)benzene; 1,2-bis(di-adamantylphosphinomethyl)-4-t-butyl benzene; 1-(P,P adamantyl, t-butyl phosphinomethyl)-2-(di-t-butylphosphinomethyl)-4,5-di-(2′-phenylprop-2′-yl)benzene; 1-(P,P adamantyl, t-butyl phosphinomethyl)-2-(di-t-butylphosphinomethyl)-4-(2′-phenylprop-2′-yl)benzene; 1-(P,P adamantyl, t-butyl phosphinomethyl)-2-(di-t-butylphosphinomethyl)-4,5-(di-t-butyl)benzene; 1-(P,P adamantyl, t-butyl phosphinomethyl)-2-(di-t-butylphosphinomethyl)-4-t-butylbenzene; 1-(2-phosphinomethyl-1,3,5,7-tetramethyl-6,9,10-trioxa-adamantyl)-2-(di-t-butylphosphinomethyl)-4,5-di-(2′-phenylprop-2′-yl)benzene; 1-(2-phosphinomethyl-1,3,5,7-tetramethyl-6,9,10-trioxa-adamantyl)-2-(di-t-butylphosphinomethyl)-4-(2′-phenylprop-2′-yl)benzene; 1-(2-phosphinomethyl-1,3,5,7-tetramethyl-6,9,10-trioxa-adamantyl)-2-(di-t-butylphosphinomethyl)-4,5-(di-t-butyl)benzene; 1-(2-phosphinomethyl-1,3,5,7-tetramethyl-6,9,10-trioxa-adamantyl)-2-(di-t-butylphosphinomethyl)-4-t-butyl benzene; 1-(2-phosphinomethyl-1,3,5,7-tetramethyl-6,9,10-trioxa-adamantyl)-2-(diadamantylphosphinomethyl)-4,5-di-(2′-phenylprop-2′-yl)benzene; 1-(2-phosphinomethyl-1,3,5,7-tetramethyl-6,9,10-trioxa-adamantyl)-2-(diadamantylphosphinomethyl)-4-(2′-phenylprop-2′-yl)benzene; 1-(2-phosphinomethyl-1,3,5,7-tetramethyl-6,9,10-trioxa-adamantyl)-2-(diadamantylphosphinomethyl)-4,5-(di-t-butyl)benzene; 1-(2-phosphinomethyl-1,3,5,7-tetramethyl-6,9,10-trioxa-adamantyl)-2-(diadamantylphosphinomethyl)-4-t-butyl benzene; 1-(di-t-butylphosphinomethyl)-2-(diadamantylphosphinomethyl)-4,5-di-(2′-phenylprop-2′-yl)benzene; 1-(di-t-butylphosphinomethyl)-2-(diadamantylphosphinomethyl)-4-(2′-phenylprop-2′-yl)benzene; 1-(di-t-butylphosphinomethyl)-2-(diadamantylphosphinomethyl)-4,5-(di-t-butyl)benzene; 1-(di-t-butylphosphinomethyl)-2-(diadamantylphosphinomethyl)-4-t-butyl benzene; 1,2-bis(2-phosphinomethyl-1,3,5-trimethyl-6,9,10-trioxatricyclo-{3.3.1.1[3.7]}decyl)-4,5-di-(2′-phenylprop-2′-yl)benzene; 1,2-bis(2-phosphinomethyl-1,3,5-trimethyl-6,9,10-trioxatricyclo-{3.3.1.1[3.7]}decyl)-4-(2′-phenylprop-2′-yl)benzene; 1,2-bis(2-phosphinomethyl-1,3,5-trimethyl-6,9,10-trioxatricyclo-{3.3.1.1[3.7]}decyl)-4,5-(di-t-butyl)benzene; 1,2-bis(2-phosphinomethyl-1,3,5-trimethyl-6,9,10-trioxatricyclo-{3.3.1.1[3.7]}decyl)-4-t-butyl benzene; 1-(2-phosphinomethyl-1,3,5-trimethyl-6,9,10-trioxatricyclo-{3.3.1.1[3.7]}decyl)-2-(di-t-butylphosphinomethyl)-4,5-di-(2′-phenylprop-2′-yl)benzene; 1-(2-phosphinomethyl-1,3,5-trimethyl-6,9,10-trioxatricyclo-{3.3.1.1[3.7]}decyl)-2-(di-t-butylphosphinomethyl)-4-(2′-phenylprop-2′-yl)benzene; 1-(2-phosphinomethyl-1,3,5-trimethyl-6,9,10-trioxatricyclo-{3.3.1.1[3.7]}decyl)-2-(di-t-butylphosphinomethyl)-4,5-(di-t-butyl)benzene; 1-(2-phosphinomethyl-1,3,5-trimethyl-6,9,10-trioxatricyclo-{3.3.1.1[3.7]}decyl)-2-(di-t-butylphosphinomethyl)-4-t-butyl benzene; 1-(2-phosphinomethyl-1,3,5-trimethyl-6,9,10-trioxatricyclo-{3.3.1.1[3.7]}decyl)-2-(diadamantylphosphinomethyl)-4,5-di-(2′-phenylprop-2′-yl)benzene; 1-(2-phosphinomethyl-1,3,5-trimethyl-6,9,10-trioxatricyclo-{3.3.1.1[3.7]}decyl)-2-(diadamantylphosphinomethyl)-4-(2′-phenylprop-2′-yl)benzene; 1-(2-phosphinomethyl-1,3,5-trimethyl-6,9,10-trioxatricyclo-{3.3.1.1[3.7]}decyl)-2-(diadamantylphosphinomethyl)-4,5-(di-t-butyl)benzene; 1-(2-phosphinomethyl-1,3,5-trimethyl-6,9,10-trioxatricyclo-{3.3.1.1[3.7]}decyl)-2-(diadamantylphosphinomethyl)-4-t-butyl benzene; 1,2-bis-perfluoro(2-phosphinomethyl-1,3,5,7-tetramethyl-6,9,10-trioxatricyclo{3.3.1.1[3.7]}-decyl)-4,5-di-(2′-phenylprop-2′-yl)benzene; 1,2-bis-perfluoro(2-phosphinomethyl-1,3,5,7-tetramethyl-6,9,10-trioxatricyclo{3.3.1.1[3.7]}decyl)-4-(2′-phenylprop-2′-yl)benzene; 1,2-bis-perfluoro(2-phosphinomethyl-1,3,5,7-tetramethyl-6,9,10-trioxatricyclo{3.3.1.1[3.7]}-decyl)-4,5-(di-t-butyl)benzene; 1,2-bis-perfluoro(2-phosphinomethyl-1,3,5,7-tetramethyl-6,9,10-trioxatricyclo{3.3.1.1[3.7]}decyl)-4-t-butyl benzene; 1,2-bis-(2-phosphinomethyl-1,3,5,7-tetra(trifluoro-methyl)-6,9,10-trioxatricyclo{3.3.1.1[3.7]}decyl)-4,5-di-(2′-phenylprop-2′-yl) benzene; 1,2-bis-(2-phosphinomethyl-1,3,5,7-tetra(trifluoro-methyl)-6,9,10-trioxatricyclo{3.3.1.1[3.7]}decyl)-4-(2′-phenylprop-2′-yl)benzene; 1,2-bis-(2-phosphinomethyl-1,3,5,7-tetra(trifluoro-methyl)-6,9,10-trioxatricyclo{3.3.1.1[3.7]}decyl)-4,5-(di-t-butyl)benzene; 1,2-bis-(2-phosphinomethyl-1,3,5,7-tetra(trifluoro-methyl)-6,9,10-trioxatricyclo{3.3.1.1[3.7]}decyl)-4-t-butyl benzene, 1-(di-tert-butylphosphinomethyl)-2-(phospha-adamantyl)-4-(trimethylsilyl)benzene, 1-(diadamantylphosphinomethyl)-2-(phospha-adamantyl)-4-(trimethylsilyl)benzene, 1-(phospha-adamantyl)-2-(phospha-adamantyl)-4-(trimethylsilyl)methylbenzene, 1-(di-tert-butylphosphinomethyl)-2-(di-tert-butylphosphino)-4-(trimethylsilyl)benzene, 1-(diadamantylphosphinomethyl)-2-(diadamantylphosphino)-4-(trimethylsilyl)benzene, 1-(di-tert-butylphosphinomethyl)-2-(diadamantylphosphino)-4-(trimethylsilyl)benzene, 1-(di-tert-butylphosphinomethyl)-2-(P-(2,2,6,6-tetramethyl-phospha-cyclohexan-4-one)-4-(trimethylsilyl)benzene, 1-(di-tert-butylphosphinomethyl)-2-(P-(2,2,6,6-tetramethyl-phospha-cyclohexan-4-one)-4-(trimethylsilyl)benzene, 1-(2-(P-(2,2,6,6-tetramethyl-phospha-cyclohexan-4-one))-4-trimethylsilylbenzyl)-2,2,6,6-tetramethyl-phospha-cyclohexan-4-one, 1-(tert-butyl,adamantylphosphino)-2-(di-adamantylphosphinomethyl)-4-(trimethylsilyl)benzene- and wherein “phospha-adamantyl” is selected from 2-phospha-1,3,5,7-tetramethyl-6,9,10-trioxadamantyl,2-phospha-1,3,5-trimethyl-6,9,10 trioxadamantyl, 2-phospha-1,3,5,7-tetra(trifluoromethyl)-6,9,10-trioxadamantyl or 2-phospha-1,3,5-tri(trifluoromethyl)-6,9,10-trioxadamantyl-, 1-(ditertbutylphosphinomethyl)-2-(P-(2,2,6,6-tetramethyl-phospha-cyclohexan-4-one))-4-(trimethylsilyl)ferrocene, 1,2-bis(di-t-butylphosphinomethyl)-4,5-diphenyl ferrocene; 1,2-bis(di-t-butylphosphinomethyl)-4-(or 1′)phenylferrocene; 1,2-bis(di-t-butylphosphinomethyl)-4,5-bis-(trimethylsilyl) ferrocene; 1,2-bis(di-t-butylphosphinomethyl)-4-(or 1′)(trimethylsilyl)ferrocene; 1,2-bis(2-phosphinomethyl-1,3,5,7-tetramethyl-6,9,10-trioxa-adamantyl)-4,5-diphenylferrocene; 1,2-bis(2-phosphinomethyl-1,3,5,7-tetramethyl-6,9,10-trioxa-adamantyl) 4-(or 1′)phenylferrocene; 1,2-bis(2-phosphinomethyl-1,3,5,7-tetramethyl-6,9,10-trioxa-adamantyl)-4,5-bis-(trimethylsilyl)ferrocene; 1,2-bis(2-phosphinomethyl-1,3,5,7-tetramethyl-6,9,10-trioxa-adamantyl) 4-(or 1′)(trimethylsilyl)ferrocene; 1,2-bis(di-adamantylphosphinomethyl)-4,5 diphenylferrocene; 1,2-bis(di-adamantylphosphinomethyl)-4-(or 1′)phenyl ferrocene; 1,2-bis(di-adamantylphosphinomethyl)-4,5 bis-(trimethylsilyl)ferrocene; 1,2-bis(di-adamantylphosphinomethyl)-4-(or 1′)(trimethylsilyl) ferrocene; 1-(P,P adamantyl, t-butyl phosphinomethyl)-2-(di-t-butylphosphinomethyl)-4,5-diphenylferrocene; 1-(P,P adamantyl, t-butyl phosphinomethyl)-2-(di-t-butylphosphinomethyl)-4-(or 1′)phenylferrocene; 1-(P,P adamantyl, t-butyl phosphinomethyl)-2-(di-t-butylphosphinomethyl)-4,5-bis-(trimethylsilyl)ferrocene; 1-(P,P adamantyl, t-butyl phosphinomethyl)-2-(di-t-butylphosphinomethyl)-4-(or 1′)(trimethylsilyl)ferrocene; 1-(2-phosphinomethyl-1,3,5,7-tetramethyl-6,9,10-trioxa-adamantyl)-2-(di-t-butylphosphinomethyl)-4,5-diphenylferrocene; 1-(2-phosphinomethyl-1,3,5,7-tetramethyl-6,9,10-trioxa-adamantyl)-2-(di-t-butylphosphinomethyl)-4-(or 1′)phenyl ferrocene; 1-(2-phosphinomethyl-1,3,5,7-tetramethyl-6,9,10-trioxa-adamantyl)-2-(di-t-butylphosphinomethyl)-4,5-bis-(trimethylsilyl)ferrocene; 1-(2-phosphinomethyl-1,3,5,7-tetramethyl-6,9,10-trioxa-adamantyl)-2-(di-t-butylphosphinomethyl)-4-(or 1′)(trimethylsilyl) ferrocene; 1-(2-phosphinomethyl-1,3,5,7-tetramethyl-6,9,10-trioxa-adamantyl)-2-(diadamantylphosphinomethyl)-4,5-diphenyl ferrocene; 1-(2-phosphinomethyl-1,3,5,7-tetramethyl-6,9,10-trioxa-adamantyl)-2-(diadamantylphosphinomethyl)-4-(or 1′)phenyl ferrocene; 1-(2-phosphinomethyl-1,3,5,7-tetramethyl-6,9,10-trioxa-adamantyl)-2-(diadamantylphosphinomethyl)-4,5-bis-(trimethylsilyl) ferrocene; 1-(2-phosphinomethyl-1,3,5,7-tetramethyl-6,9,10-trioxa-adamantyl)-2-(diadamantylphosphinomethyl)-4-(or 1′) (trimethylsilyl) ferrocene; 1-(di-t-butylphosphinomethyl)-2-(diadamantylphosphinomethyl)-4,5-diphenyl ferrocene; 1-(di-t-butylphosphinomethyl)-2-(diadamantylphosphinomethyl)-4-(or 1′)phenyl ferrocene; 1-(di-t-butylphosphinomethyl)-2-(diadamantylphosphinomethyl)-4,5-bis-(trimethylsilyl) ferrocene; 1-(di-t-butylphosphinomethyl)-2-(diadamantylphosphinomethyl)-4-(or 1′)(trimethylsilyl) ferrocene; 1,2-bis(2-phosphinomethyl-1,3,5-trimethyl-6,9,10-trioxatricyclo-{3.3.1.1[3.7]}decyl)-4,5-diphenyl ferrocene; 1,2-bis(2-phosphinomethyl-1,3,5-trimethyl-6,9,10-trioxatricyclo-{3.3.1.1[3.7]}decyl)-4-(or 1′)phenyl ferrocene; 1,2-bis(2-phosphinomethyl-1,3,5-trimethyl-6,9,10-trioxatricyclo-{3.3.1.1[3.7]}decyl)-4,5-bis-(trimethylsilyl) ferrocene; 1,2-bis(2-phosphinomethyl-1,3,5-trimethyl-6,9,10-trioxatricyclo-{3.3.1.1[3.7]}decyl)-4-(or 1′)(trimethylsilyl) ferrocene; 1-(2-phosphinomethyl-1,3,5-trimethyl-6,9,10-trioxatricyclo-{3.3.1.1[3.7]}decyl)-2-(di-t-butylphosphinomethyl)-4,5-diphenyl ferrocene; 1-(2-phosphinomethyl-1,3,5-trimethyl-6,9,10-trioxatricyclo-{3.3.1.1[3.7]}decyl)-2-(di-t-butylphosphinomethyl)-4-(or 1′)phenyl ferrocene; 1-(2-phosphinomethyl-1,3,5-trimethyl-6,9,10-trioxatricyclo-{3.3.1.1[3.7]}decyl)-2-(di-t-butylphosphinomethyl)-4,5-bis-(trimethylsilyl) ferrocene; 1-(2-phosphinomethyl-1,3,5-trimethyl-6,9,10-trioxatricyclo-{3.3.1.1[3.7]}decyl)-2-(di-t-butylphosphinomethyl)-4-(or 1′) (trimethylsilyl) ferrocene; 1-(2-phosphinomethyl-1,3,5-trimethyl-6,9,10-trioxatricyclo-{3.3.1.1[3.7]}decyl)-2-(diadamantylphosphinomethyl)-4,5-diphenyl ferrocene; 1-(2-phosphinomethyl-1,3,5-trimethyl-6,9,10-trioxatricyclo-{3.3.1.1[3.7]}decyl)-2-(diadamantylphosphinomethyl)-4-(or 1′)phenyl ferrocene; 1-(2-phosphinomethyl-1,3,5-trimethyl-6,9,10-trioxatricyclo-{3.3.1.1[3.7]}decyl)-2-(diadamantylphosphinomethyl)-4,5-bis-(trimethylsilyl) ferrocene; 1-(2-phosphinomethyl-1,3,5-trimethyl-6,9,10-trioxatricyclo-{3.3.1.1[3.7]}decyl)-2-(diadamantylphosphinomethyl)-4-(or 1′)(trimethylsilyl) ferrocene; 1,2-bis-perfluoro(2-phosphinomethyl-1,3,5,7-tetramethyl-6,9,10-trioxatricyclo{3.3.1.1[3.7]}-decyl)-4,5-diphenyl ferrocene; 1,2-bis-perfluoro(2-phosphinomethyl-1,3,5,7-tetramethyl-6,9,10-trioxatricyclo{3.3.1.1[3.7]}decyl)-4-(or 1′)phenyl ferrocene; 1,2-bis-perfluoro(2-phosphinomethyl-1,3,5,7-tetramethyl-6,9,10-trioxatricyclo{3.3.1.1[3.7]}-decyl)-4,5-bis-(trimethylsilyl) ferrocene; 1,2-bis-perfluoro(2-phosphinomethyl-1,3,5,7-tetramethyl-6,9,10-trioxatricyclo{3.3.1.1[3.7]}decyl)-4-(or 1′)(trimethylsilyl) ferrocene; 1,2-bis-(2-phosphinomethyl-1,3,5,7-tetra(trifluoro-methyl)-6,9,10-trioxatricyclo{3.3.1.1[3.7]}decyl)-4,5-diphenyl ferrocene; 1,2-bis-(2-phosphinomethyl-1,3,5,7-tetra(trifluoro-methyl)-6,9,10-trioxatricyclo{3.3.1.1[3.7]}decyl)-4-(or 1′)phenyl ferrocene; 1,2-bis-(2-phosphinomethyl-1,3,5,7-tetra(trifluoro-methyl)-6,9,10-trioxatricyclo{3.3.1.1[3.7]}decyl)-4,5-bis-(trimethylsilyl) ferrocene; 1,2-bis-(2-phosphinomethyl-1,3,5,7-tetra(trifluoro-methyl)-6,9,10-trioxatricyclo{3.3.1.1[3.7]}decyl)-4-(or 1′)(trimethylsilyl) ferrocene; 1,2-bis(di-t-butylphosphinomethyl)-4,5-di-(2′-phenylprop-2′-yl)ferrocene; 1,2-bis(di-t-butylphosphinomethyl)-4-(or 1′)(2′-phenylprop-2′-yl)ferrocene; 1,2-bis(di-t-butylphosphinomethyl)-4,5-di-t-butyl ferrocene; 1,2-bis(di-t-butylphosphinomethyl)-4-(or 1′)ₜ-butylferrocene; 1,2-bis(2-phosphinomethyl-1,3,5,7-tetramethyl-6,9,10-trioxa-adamantyl)-4,5-di-(2′-phenylprop-2′-yl)ferrocene; 1,2-bis(2-phosphinomethyl-1,3,5,7-tetramethyl-6,9,10-trioxa-adamantyl)-4-(or 1′)(2′-phenylprop-2′-yl)ferrocene; 1,2-bis(2-phosphinomethyl-1,3,5,7-tetramethyl-6,9,10-trioxa-adamantyl)-4,5-(di-t-butyl)ferrocene; 1,2-bis(2-phosphinomethyl-1,3,5,7-tetramethyl-6,9,10-trioxa-adamantyl)-4-(or 1′)ₜ-butylferrocene; 1,2-bis(di-adamantylphosphinomethyl)-4,5-di-(2′-phenylprop-2′-yl) ferrocene; 1,2-bis(di-adamantylphosphinomethyl)-4-(or 1′)(2′-phenylprop-2′-yl) ferrocene; 1,2-bis(di-adamantylphosphinomethyl)-4,5-(di-t-butyl) ferrocene; 1,2-bis(di-adamantylphosphinomethyl)-4-(or 1′)ₜ-butyl ferrocene; 1-(P,P adamantyl, t-butyl phosphinomethyl)-2-(di-t-butylphosphinomethyl)-4,5-di-(2′-phenylprop-2′-yl)ferrocene; 1-(P,P adamantyl, t-butyl phosphinomethyl)-2-(di-t-butylphosphinomethyl)-4-(or 1′)(2′-phenylprop-2′-yl)ferrocene; 1-(P,P adamantyl, t-butyl phosphinomethyl)-2-(di-t-butylphosphinomethyl)-4,5-(di-t-butyl)ferrocene; 1-(P,P adamantyl, t-butyl phosphinomethyl)-2-(di-t-butylphosphinomethyl)-4-(or 1′)ₜ-butylferrocene; 1-(2-phosphinomethyl-1,3,5,7-tetramethyl-6,9,10-trioxa-adamantyl)-2-(di-t-butylphosphinomethyl)-4,5-di-(2′-phenylprop-2′-yl)ferrocene; 1-(2-phosphinomethyl-1,3,5,7-tetramethyl-6,9,10-trioxa-adamantyl)-2-(di-t-butylphosphinomethyl)-4-(or 1′)(2′-phenylprop-2′-yl) ferrocene; 1-(2-phosphinomethyl-1,3,5,7-tetramethyl-6,9,10-trioxa-adamantyl)-2-(di-t-butylphosphinomethyl)-4,5-(di-t-butyl)ferrocene; 1-(2-phosphinomethyl-1,3,5,7-tetramethyl-6,9,10-trioxa-adamantyl)-2-(di-t-butylphosphinomethyl)-4-(or 1′)ₜ-butyl ferrocene; 1-(2-phosphinomethyl-1,3,5,7-tetramethyl-6,9,10-trioxa-adamantyl)-2-(diadamantylphosphinomethyl)-4,5-di-(2′-phenylprop-2′-yl) ferrocene; 1-(2-phosphinomethyl-1,3,5,7-tetramethyl-6,9,10-trioxa-adamantyl)-2-(diadamantylphosphinomethyl)-4-(or 1′)(2′-phenylprop-2′-yl) ferrocene; 1-(2-phosphinomethyl-1,3,5,7-tetramethyl-6,9,10-trioxa-adamantyl)-2-(diadamantylphosphinomethyl)-4,5-(di-t-butyl) ferrocene; 1-(2-phosphinomethyl-1,3,5,7-tetramethyl-6,9,10-trioxa-adamantyl)-2-(diadamantylphosphinomethyl)-4-(or 1′)ₜ-butyl ferrocene; 1-(di-t-butylphosphinomethyl)-2-(diadamantylphosphinomethyl)-4,5-di-(2′-phenylprop-2′-yl) ferrocene; 1-(di-t-butylphosphinomethyl)-2-(diadamantylphosphinomethyl)-4-(or 1′)(2′-phenylprop-2′-yl) ferrocene; 1-(di-t-butylphosphinomethyl)-2-(diadamantylphosphinomethyl)-4,5-(di-t-butyl) ferrocene; 1-(di-t-butylphosphinomethyl)-2-(diadamantylphosphinomethyl)-4-(or 1′)ₜ-butyl ferrocene; 1,2-bis(2-phosphinomethyl-1,3,5-trimethyl-6,9,10-trioxatricyclo-{3.3.1.1[3.7]}decyl)-4,5-di-(2′-phenylprop-2′-yl) ferrocene; 1,2-bis(2-phosphinomethyl-1,3,5-trimethyl-6,9,10-trioxatricyclo-{3.3.1.1[3.7]}decyl)-4-(or 1′)(2′-phenylprop-2′-yl) ferrocene; 1,2-bis(2-phosphinomethyl-1,3,5-trimethyl-6,9,10-trioxatricyclo-{3.3.1.1[3.7]}decyl)-4,5-(di-t-butyl) ferrocene; 1,2-bis(2-phosphinomethyl-1,3,5-trimethyl-6,9,10-trioxatricyclo-{3.3.1.1[3.7]}decyl)-4-(or 1′)ₜ-butyl ferrocene; 1-(2-phosphinomethyl-1,3,5-trimethyl-6,9,10-trioxatricyclo-{3.3.1.1[3.7]}decyl)-2-(di-t-butylphosphinomethyl)-4,5-di-(2′-phenylprop-2′-yl) ferrocene; 1-(2-phosphinomethyl-1,3,5-trimethyl-6,9,10-trioxatricyclo-{3.3.1.1[3.7]}decyl)-2-(di-t-butylphosphinomethyl)-4-(or 1′)(2′-phenylprop-2′-yl) ferrocene; 1-(2-phosphinomethyl-1,3,5-trimethyl-6,9,10-trioxatricyclo-{3.3.1.1[3.7]}decyl)-2-(di-t-butylphosphinomethyl)-4,5-(di-t-butyl) ferrocene; 1-(2-phosphinomethyl-1,3,5-trimethyl-6,9,10-trioxatricyclo-{3.3.1.1[3.7]}decyl)-2-(di-t-butylphosphinomethyl)-4-(or 1′)ₜ-butyl ferrocene; 1-(2-phosphinomethyl-1,3,5-trimethyl-6,9,10-trioxatricyclo-{3.3.1.1[3.7]}decyl)-2-(diadamantylphosphinomethyl)-4,5-di-(2′-phenylprop-2′-yl) ferrocene; 1-(2-phosphinomethyl-1,3,5-trimethyl-6,9,10-trioxatricyclo-{3.3.1.1[3.7]}decyl)-2-(diadamantylphosphinomethyl)-4-(or 1′)(2′-phenylprop-2′-yl) ferrocene; 1-(2-phosphinomethyl-1,3,5-trimethyl-6,9,10-trioxatricyclo-{3.3.1.1[3.7]}decyl)-2-(diadamantylphosphinomethyl)-4,5-(di-t-butyl) ferrocene; 1-(2-phosphinomethyl-1,3,5-trimethyl-6,9,10-trioxatricyclo-{3.3.1.1[3.7]}decyl)-2-(diadamantylphosphinomethyl)-4-(or 1′)ₜ-butyl ferrocene; 1,2-bis-perfluoro(2-phosphinomethyl-1,3,5,7-tetramethyl-6,9,10-trioxatricyclo{3.3.1.1[3.7]}-decyl)-4,5-di-(2′-phenylprop-2′-yl) ferrocene; 1,2-bis-perfluoro(2-phosphinomethyl-1,3,5,7-tetramethyl-6,9,10-trioxatricyclo{3.3.1.1[3.7]}decyl)-4-(or 1′)(2′-phenylprop-2′-yl) ferrocene; 1,2-bis-perfluoro(2-phosphinomethyl-1,3,5,7-tetramethyl-6,9,10-trioxatricyclo{3.3.1.1[3.7]}-decyl)-4,5-(di-t-butyl) ferrocene; 1,2-bis-perfluoro(2-phosphinomethyl-1,3,5,7-tetramethyl-6,9,10-trioxatricyclo{3.3.1.1[3.7]}decyl)-4-(or 1′)ₜ-butyl ferrocene; 1,2-bis-(2-phosphinomethyl-1,3,5,7-tetra(trifluoro-methyl)-6,9,10-trioxatricyclo{3.3.1.1[3.7]}decyl)-4,5-di-(2′-phenylprop-2′-yl) ferrocene; 1,2-bis-(2-phosphinomethyl-1,3,5,7-tetra(trifluoro-methyl)-6,9,10-trioxatricyclo{3.3.1.1[3.7]}decyl)-4-(or 1′)(2′-phenylprop-2′-yl) ferrocene; 1,2-bis-(2-phosphinomethyl-1,3,5,7-tetra(trifluoro-methyl)-6,9,10-trioxatricyclo{3.3.1.1[3.7]}decyl)-4,5-(di-t-butyl) ferrocene; 1,2-bis-(2-phosphinomethyl-1,3,5,7-tetra(trifluoro-methyl)-6,9,10-trioxatricyclo{3.3.1.1[3.7]}decyl)-4-(or 1′)ₜ-butyl ferrocene.

Selected structures of ligands of the invention include:—

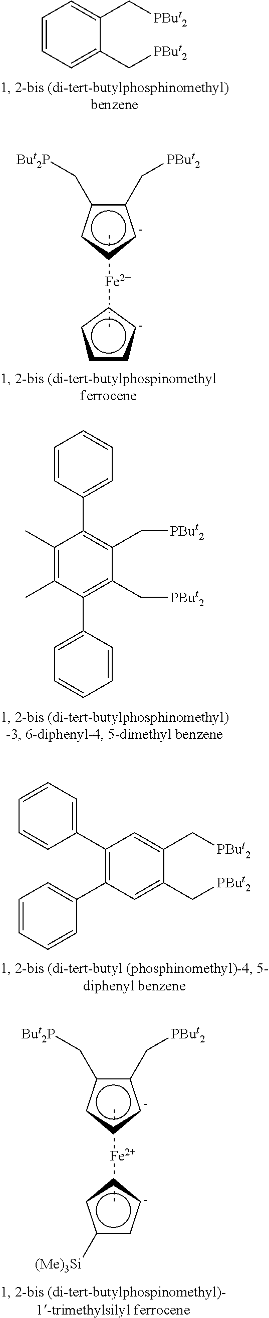

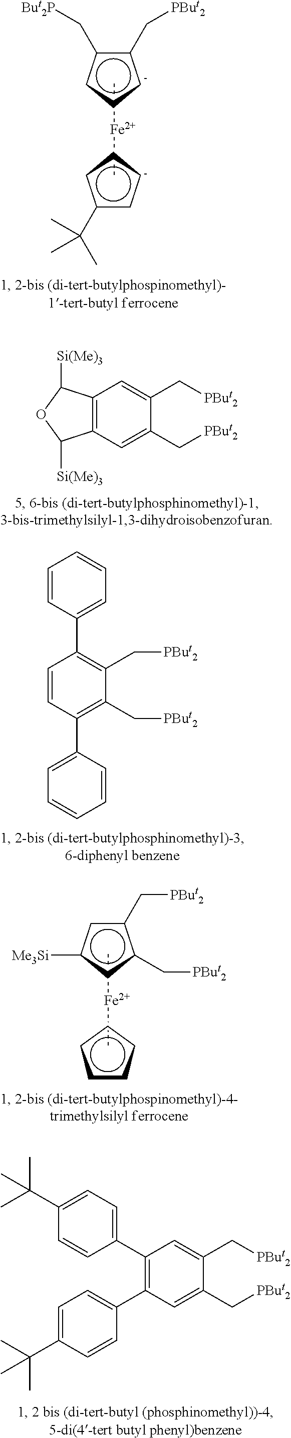

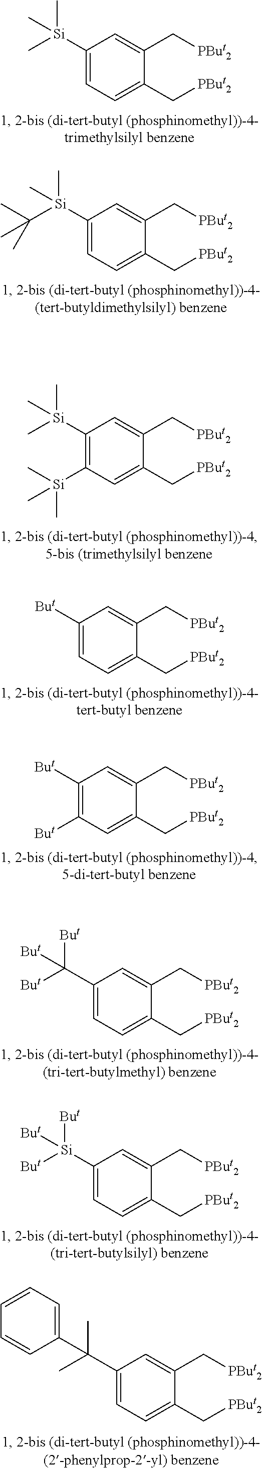

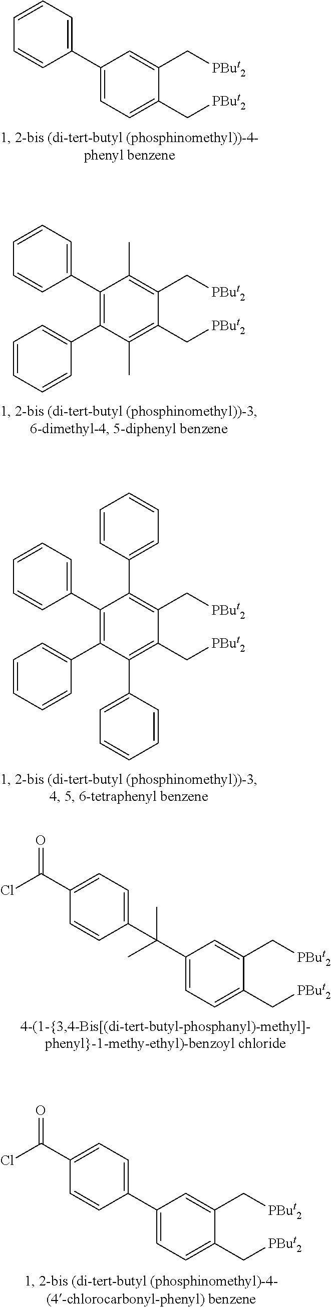

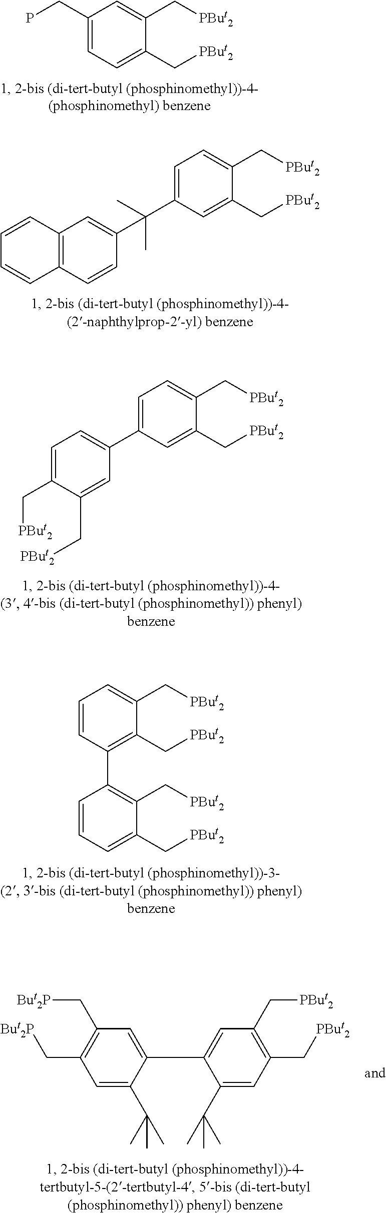

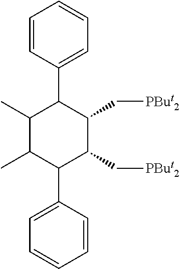

### cis-1,2-bis(di-tert-butylphosphinomethyl), 3,6, diphenyl-4,5 dimethyl-cyclohexane

Examples of norbornyl bridge non-aromatic bridged ligands include:—

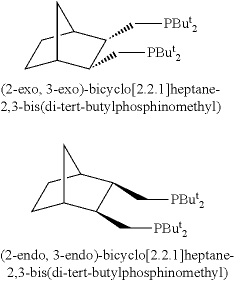

Examples of substituted non-aromatic bridged ligand structures include:—

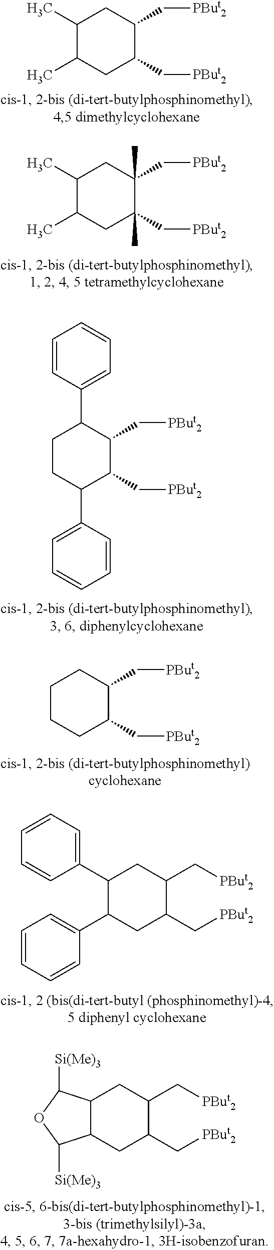

In the above example, structures of ligands of general formula (I), one or more of the X¹-X⁴ tertiary carbon bearing groups, t-butyl, attached to the Q¹ and/or Q² group phosphorus may be replaced by a suitable alternative. Preferred alternatives are adamantyl, 1,3 dimethyl adamantyl, congressyl, norbornyl or 1-norbondienyl, or X¹ and X² together and/or X³ and X⁴ together form together with the phosphorus a 2-phospha-tricyclo[3.3.1.1{3,7} decyl group or a ring system of formula 1a or 1b as defined herein such as 2-phospha-1,3,5,7-tetramethyl-6,9,10-trioxadamantyl, 2-phospha-1,3,5-trimethyl-6,9,10-trioxadamantyl or P-2,2,6,6-tetramethyl-phospha-cyclohexan-4-one. In most embodiments, it is preferred that the X¹-X⁴ groups or the combined X¹/X² and X³/X⁴ groups are the same but it may also be advantageous to use different groups to produce asymmetry around the active site in these selected ligands and generally in this invention.

Typically, the group X¹ represents CR¹(R²)(R³), X² represents CR⁴ (R⁵)(R⁶), X³ represents CR⁷(R⁸)(R⁹) and X⁴ represents CR¹⁰(R¹¹)(R¹²), wherein R¹ to R¹² represent alkyl, aryl or het.

Particularly preferred is when the organic groups R¹-R³, R⁴-R⁶, R⁷-R⁹ and/or R¹⁰-R¹² or, alternatively, R¹-R⁶ and/or R⁷-R¹² when associated with their respective tertiary carbon atom(s) form composite groups which are at least as sterically hindering as t-butyl(s).

The steric composite groups may be cyclic, part-cyclic or acyclic. When cyclic or part cyclic, the group may be substituted or unsubstituted or saturated or unsaturated. The cyclic or part cyclic groups may preferably contain, including the tertiary carbon atom(s), from C₄-C₃₄, more preferably C₈-C₂₄, most preferably C₁₀-C₂₀ carbon atoms in the cyclic structure. The cyclic structure may be substituted by one or more substituents selected from halo, cyano, nitro, OR¹⁹, OC(O)R²⁰, C(O)R²¹, C(O)OR²², NR²³R²⁴, C(O)NR²⁵R²⁶, SR²⁹, C(O)SR³⁰, C(S)NR²⁷R²⁸, aryl or Het, wherein R¹⁹ to R³⁰ are as defined herein, and/or be interrupted by one or more oxygen or sulphur atoms, or by silano or dialkylsilicon groups.

In particular, when cyclic, X¹, X², X³ and/or X⁴ may represent congressyl, norbornyl, 1-norbornadienyl or adamantyl, or X¹ and X² together with Q² to which they are attached form an optionally substituted 2-Q²-tricyclo[3.3.1.1{3,7}]decyl group or derivative thereof, or X¹ and X² together with Q² to which they are attached form a ring system of formula 1a

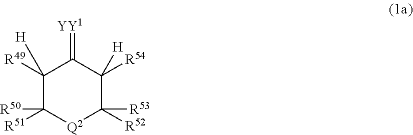

Similarly, X³ and X⁴ together with Q¹ to which they are attached may form an optionally substituted 2-Q1-tricyclo[3.3.1.1{3,7}]decyl group or derivative thereof, or X³ and X⁴ together with Q¹ to which they are attached may form a ring system of formula 1b

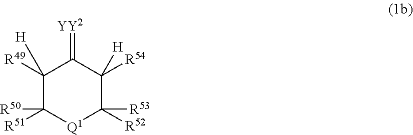

Alternatively, one or more of the groups X¹, X², X³ and/or X⁴ may represent a solid phase to which the ligand is attached.

Particularly preferred is when X¹, X², X³ and X⁴ or X¹ and X² together with its respective Q² atom and X³ and X⁴ together with its respective Q¹ atom are the same or when X¹ and X³ are the same whilst X² and X⁴ are different but the same as each other.

In preferred embodiments, R¹ to R¹² each independently represent alkyl, aryl, or Het;

R¹⁹ to R³⁰ each independently represent hydrogen, alkyl, aryl or Het;

R⁴⁹ and R⁵⁴, when present, each independently represent hydrogen, alkyl or aryl;

R⁵⁰ to R⁵³, when present, each independently represent alkyl, aryl or Het;

YY¹ and YY², when present, each independently represent oxygen, sulfur or N—R⁵⁵, wherein R⁵⁵ represents hydrogen, alkyl or aryl.

An example compound of the above formulas Ia or Ib, when R⁵⁰-R⁵³ are methyl, R⁴⁹ and R⁵⁴ are H, YY¹ or YY² are 0 and Q¹ or Q² are phosphorus is 2,2,6,6-tetramethyl-phospha-cyclohexan-4-one.

Preferably, R¹ to R¹² each independently represent alkyl or aryl. More preferably, R¹ to R¹² each independently represent C₁ to C₆ alkyl, C₁-C₆ alkyl phenyl (wherein the phenyl group is optionally substituted as aryl as defined herein) or phenyl (wherein the phenyl group is optionally substituted as aryl as defined herein). Even more preferably, R¹ to R¹² each independently represent C₁ to C₆ alkyl, which is optionally substituted as alkyl as defined herein. Most preferably, R¹ to R¹² each represent non-substituted C₁ to C₆ alkyl such as methyl, ethyl, n-propyl, iso-propyl, n-butyl, iso-butyl, tert-butyl, pentyl, hexyl and cyclohexyl, especially methyl.

In a particularly preferred embodiment of the present invention R¹, R⁴, R⁷ and R¹⁰ each represent the same alkyl, aryl or Het moiety as defined herein, R²R⁵, R⁸ and R¹¹ each represent the same alkyl, aryl or Het moiety as defined herein, and R³, R⁶, R⁹ and R¹² each represent the same alkyl, aryl or Het moiety as defined herein. More preferably R¹, R⁴, R⁷ and R¹⁰ each represent the same C₁-C₆ alkyl, particularly non-substituted C₁-C₆ alkyl, such as methyl, ethyl, n-propyl, iso-propyl, n-butyl, iso-butyl, tert-butyl, pentyl, hexyl or cyclohexyl; R², R⁵, R⁸ and R¹¹ each independently represent the same C₁-C₆ alkyl as defined above; and R³, R⁶, R⁹ and R¹² each independently represent the same C₁-C₆ alkyl as defined above. For example: R¹, R⁴, R⁷ and R¹⁰ each represent methyl; R², R⁵, R⁸ and R¹¹ each represent ethyl; and, R³, R⁶, R⁹ and R¹² each represent n-butyl or n-pentyl.

In an especially preferred embodiment of the present invention each R¹ to R¹² group represents the same alkyl, aryl, or Het moiety as defined herein. Preferably, when alkyl groups, each R¹ to R¹² represents the same C₁ to C₆ alkyl group, particularly non-substituted C₁-C₆ alkyl, such as methyl, ethyl, n-propyl, iso-propyl, n-butyl, iso-butyl, tert-butyl, pentyl, hexyl and cyclohexyl. More preferably, each R¹ to R¹² represents methyl or tert-butyl, most preferably, methyl.

The 2-Q² (or Q¹)-tricyclo[3.3.1.1.{3,7}]decyl group (referred to hereinafter as a 2-meta-adamantyl group for convenience wherein 2-meta-adamantyl is a reference to Q¹ or Q² being an arsenic, antimony or phosphorus atom i.e. 2-arsa-adamantyl and/or 2-stiba-adamantyl and/or 2-phospha-adamantyl, preferably, 2-phospha-adamantyl) may optionally comprise, beside hydrogen atoms, one or more substituents. Suitable substituents include those substituents as defined herein in respect of the adamantyl group. Highly preferred substituents include alkyl, particularly unsubstituted C₁-C₈ alkyl, especially methyl, trifluoromethyl, —OR¹⁹ wherein R¹⁹ is as defined herein particularly unsubstituted C₁-C₈ alkyl or aryl, and 4-dodecylphenyl. When the 2-meta-adamantyl group includes more than one substituent, preferably each substituent is identical.

Preferably, the 2-meta-adamantyl group is substituted on one or more of the 1, 3, 5 or 7 positions with a substituent as defined herein. More preferably, the 2-meta-adamantyl group is substituted on each of the 1, 3 and 5 positions. Suitably, such an arrangement means the Q atom of the 2-meta-adamantyl group is bonded to carbon atoms in the adamantyl skeleton having no hydrogen atoms. Most preferably, the 2-meta-adamantyl group is substituted on each of the 1, 3, 5 and 7 positions. When the 2-meta-adamantyl group includes more than 1 substituent preferably each substituent is identical. Especially preferred substituents are unsubstituted C₁-C₈ alkyl and haloakyls, particularly unsubstituted C₁-C₈ alkyl such as methyl and fluorinated C₁-C₈ alkyl such as trifluoromethyl.

Preferably, 2-meta-adamantyl represents unsubstituted 2-meta-adamantyl or 2-meta-adamantyl substituted with one or more unsubstituted C₁-C₈ alkyl substituents, or a combination thereof.

Preferably, the 2-meta-adamantyl group includes additional heteroatoms, other than the 2-Q atom, in the 2-meta-adamantyl skeleton. Suitable additional heteroatoms include oxygen and sulphur atoms, especially oxygen atoms. More preferably, the 2-meta-adamantyl group includes one or more additional heteroatoms in the 6, 9 and 10 positions. Even more preferably, the 2-meta-adamantyl group includes an additional heteroatom in each of the 6, 9 and 10 positions. Most preferably, when the 2-meta-adamantyl group includes two or more additional heteroatoms in the 2-meta-adamantyl skeleton, each of the additional heteroatoms are identical. Preferably, the 2-meta-adamantyl includes one or more oxygen atoms in the 2-meta-adamantyl skeleton. An especially preferred 2-meta-adamantyl group, which may optionally be substituted with one or more substituents as defined herein, includes an oxygen atom in each of the 6, 9 and 10 positions of the 2-meta-adamantyl skeleton.

Highly preferred 2-meta-adamantyl groups as defined herein include 2-phospha-1,3,5,7-tetramethyl-6,9,10-trioxadamantyl, 2-phospha-1,3,5-trimethyl-6,9,10-trioxadamantyl, 2-phospha-1,3,5,7-tetra(trifluoromethyl)-6,9,10-trioxadamantyl group, and 2-phospha-1,3,5-tri(trifluoromethyl)-6,9,10-trioxadamantyl group. Most preferably, the 2-phospha-adamantyl is selected from 2-phospha-1,3,5,7-tetramethyl-6,9,10-trioxadamantyl group or 2-phospha-1,3,5′-trimethyl-6,9,10-trioxadamantyl group.

Preferably, when more than one 2-meta-adamantyl group is present in a compound of formula I, each 2-meta-adamantyl group is identical. However, it can also be advantageous if asymmetric ligands are prepared and if such ligands include a 2-meta-adamantyl group incorporating the Q¹ atom then other groups can be found on the Q² atom or vice versa.

The 2-meta-adamantyl group may be prepared by methods well known to those skilled in the art. Suitably, certain 2-phospha-adamantyl compounds are obtainable from Cytec Canada Inc, Canada. Likewise corresponding 2-meta-adamantyl compounds of formula I etc may be obtained from the same supplier or prepared by analogous methods.

Preferred embodiments of the present invention include those wherein:

X³ represents CR⁷(R²)(R³), X⁴ represents CR¹⁰(R¹¹)(R¹²), X¹ represents CR¹(R²)(R³) and X² represents CR⁴(R⁵)(R⁶);
X³ represents CR⁷(R⁸)(R⁹), X⁴ represents CR¹⁰(R¹¹)(R¹), and X¹ and X² together with Q² to which they are attached form a 2-phospha-adamantyl group;
X³ represents CR⁷(R⁸)(R⁹), X⁴ represents CR¹⁰(R¹¹)(R¹²); and X¹ and X² together with Q² to which they are attached form a ring system of formula 1a;

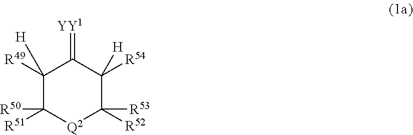

X³ represents CR⁷(R⁸)(R⁹), X⁴ represents adamantyl, and X¹ and X² together with Q² to which they are attached form a 2-phospha-adamantyl group;
X³ represents CR⁷(R⁸)(R⁹), X⁴ represents adamantyl and X¹ and X² together with Q² to which they are attached form a ring system of formula 1a;

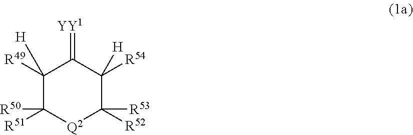

X³ represents CR⁷(R⁸)(R⁹), X⁴ represents adamantyl, X¹ represents CR¹(R²)(R³) and X² represents CR⁴(R⁵)(R⁶);
X³ represents CR⁷(R⁸)(R⁹), X⁴ represents congressyl, and X¹ and X² together with Q² to which they are attached form a 2-phospha-adamantyl group;
X³ represents CR⁷(R⁸)(R⁹), X⁴ represents congressyl, X¹ represents CR¹(R²)(R³) and X² represents CR⁴(R⁵)(R⁶);
X³ and X⁴ independently represent adamantyl, and X¹ and X² together with Q² to which they are attached form a 2-phospha-adamantyl group;
X³ and X⁴ independently represent adamantyl, and X¹ and X² together with Q² to which they are attached form a ring system of formula 1a;

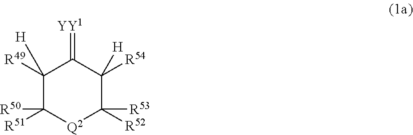

X³ and X⁴ independently represent adamantyl, X¹ represents CR¹(R²)(R³) and X² represents CR⁴(R⁵)(R⁶);
X¹, X², X³ and X⁴ represent adamantyl;
X³ and X⁴ together with Q¹ to which they are attached may form a ring system of formula 1b

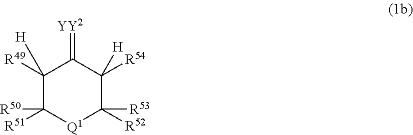

and X¹ and X² together with Q² to which they are attached form a ring system of formula 1a;

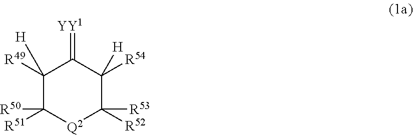

X³ and X⁴ independently represent congressyl, and X¹ and X² together with Q² to which they are attached form a 2-phospha-adamantyl group;
X³ and X⁴ together with Q¹ to which they are attached may form a ring system of formula 1b

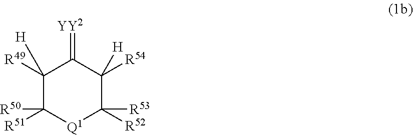

and X¹ and X² together with Q², to which they are attached form a 2-phospha-adamantyl group;
X³ and X⁴ independently represent congressyl, and X¹ represents CR¹(R²)(R³) and X² represents CR⁴(R⁵)(R⁶);
X³ and X⁴ together with Q¹ to which they are attached may form a ring system of formula 1b

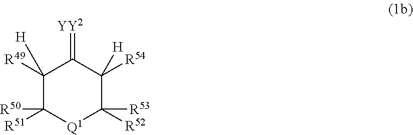

X¹ represents CR¹(R²)(R³) and X² represents CR⁴(R⁵)(R⁶);
X³ and X⁴ together with Q¹ to which they are attached form a 2-phospha-adamantyl group, and X¹ and X² together with Q² to which they are attached form a 2-phospha-adamantyl group

Highly preferred embodiments of the present invention include those wherein:

X³ represents CR⁷(R⁸) (R⁹), X⁴ represents CR¹⁰(R¹¹)(R¹²), X¹ represents CR¹(R²)(R³) and X² represents CR⁴(R⁵)(R⁶); especially where R¹-R¹² are methyl.

Preferably in a compound of formula I, X³ is identical to X⁴ and/or X¹ is identical to X².

Particularly preferred combinations in the present invention include those wherein:—
(1) X³ represents CR⁷(R⁸)(R⁹), X⁴ represents CR¹⁰(R¹¹) (R¹²), X¹ represents CR¹(R²)(R³) and X² represents CR⁴(R⁵)(R⁶);
   A and B are the same and represent —CH₂—;
   Q¹ and Q² both represent phosphorus linked to the R group at ring positions 1 and 2;
   R represents 4-(trimethylsilyl)-benzene-1,2-diyl
(2) X³ represents CR⁷(R⁸)(R⁹), X⁴ represents CR¹⁰(R¹¹)(R¹²), X¹ represents CR¹(R²)(R³) and X² represents CR⁴(R⁵)(R⁶);
   A and B are the same and represent —CH₂—;
   Q¹ and Q² both represent phosphorus linked to the R group at ring positions 1 and 2;
   R represents 4-t-butyl-benzene-1,2-diyl.
(3) X³ and X⁴ together with Q¹ to which they are attached form a 2-phospha-adamantyl group, and, X¹ and X² together with Q² to which they are attached form a 2-phospha-adamantyl group;
   A and B are the same and represent —CH₂—;
   Q¹ and Q² both represent phosphorus linked to the R group at ring positions 1 and 2;
   R represents 4-(trimethylsilyl)-benzene-1,2-diyl.
(4) X¹, X², X³ and X⁴ represent adamantyl;
   A and B are the same and represent —CH₂—;
   Q¹ and Q² both represent phosphorus linked to the R group at ring positions 1 and 2;
   R represents 4-(trimethylsilyl)-benzene-1,2-diyl.

Preferably, in the compound of formula I, A and B each independently represents C₁ to C₆ alkylene which is optionally substituted as defined herein, for example with alkyl groups. Preferably, the lower alkylene groups which A and B represent are non-substituted. Particularly preferred alkylene which A and B may independently represent are —CH₂— or —C₂H₄—. Most preferably, each of A and B represent the same alkylene as defined herein, particularly —CH₂—. Alternatively, one of A or B is C₀ ie Q² or Q¹ is connected directly to the group R and the other Q group is not connected directly to the group R and is a C₁ to C₆ alkylene, preferably —CH₂— or —C₂H₄—, most preferably, —CH₂—.

Still further preferred compounds of formula I include those wherein:

R¹ to R¹² are alkyl and are the same and preferably, each represents C₁ to C₆ alkyl, particularly methyl.

Especially preferred specific compounds of formula I include those wherein:

each R¹ to R¹² is the same and represents methyl;
A and B are the same and represent —CH₂—;
R represents 4-t-butyl-benzene-1,2-diyl or 4(trimethylsilyl)-benzene-1,2-diyl.

The term “lower alkylene” which A and B represent in a compound of formula I, when used herein, includes C₀-C₁₀ or C₁ to C₁₀ groups which, in the latter case, can be bonded at two places on the group to thereby connect the group Q¹ or Q² to the R group, and, in the latter case, is otherwise defined in the same way as “alkyl” below. Nevertheless, in the latter case, methylene is most preferred. In the former case, by C₀ is meant that the group Q¹ or Q² is connected directly to the R group and there is no C₁-C₁₀ lower alkylene group and in this case only one of A and B is a C₁-C₁₀ lower alkylene. In any case, when one of the groups A or B is C₀ then the other group cannot be Co and must be a C₁-C₁₀ group as defined herein and, therefore, at least one of A and B is a C₁-C₁₀ “lower alkylene” group.

The term “alkyl” when used herein, means C₁ to C₁₀ alkyl and includes methyl, ethyl, ethenyl, propyl, propenyl butyl, butenyl, pentyl, pentenyl, hexyl, hexenyl and heptyl groups. Unless otherwise specified, alkyl groups may, when there is a sufficient number of carbon atoms, be linear or branched (particularly preferred branched groups include t-butyl and isopropyl), be saturated or unsaturated, be cyclic, acyclic or part cyclic/acyclic, be unsubstituted, substituted or terminated by one or more substituents selected from halo, cyano, nitro, OR¹⁹, OC(O)R²⁰, C(O)R²¹, C(O)OR²², NR²³R²⁴, C(O)NR²⁵R²⁶, SR²⁹, C(O)SR³⁰, C(S)NR²⁷R²⁸, unsubstituted or substituted aryl, or unsubstituted or substituted Het and/or be interrupted by one or more (preferably less than 4) oxygen, sulphur, silicon atoms, or by silano or dialkylsilicon groups, or mixtures thereof.

R¹⁹ to R³⁰ herein each independently represent hydrogen, halo, unsubstituted or substituted aryl or unsubstituted or substituted alkyl, or, in the case of R²¹ additionally, halo, nitro, cyano, thio and amino.

The term “Ar” or “aryl” when used herein, includes five-to-ten-membered, preferably five to eight membered, carbocyclic aromatic or pseudo aromatic groups, such as phenyl, cyclopentadienyl and indenyl anions and naphthyl, which groups may be unsubstituted or as one option substituted with one or more substituents selected from unsubstituted or substituted aryl, alkyl (which group may itself be unsubstituted or substituted or terminated as defined herein), Het (which group may itself be unsubstituted or substituted or terminated as defined herein), halo, cyano, nitro, OR¹⁹, OC(O)R²⁰, C(O)R²¹, C(O)OR²², NR²³R²⁴, C(O)NR²⁵R²⁶, SR²⁹, C(O)SR³⁰ or C(S)NR²⁷R²⁸ wherein R¹⁹ to R³⁰ are as defined herein.

The term “alkenyl” when used herein, means C₂ to C₁₀ alkenyl and includes ethenyl, propenyl, butenyl, pentenyl, and hexenyl groups. Unless otherwise specified, alkenyl groups may, when there is a sufficient number of carbon atoms, be linear or branched, be saturated or unsaturated, be cyclic, acyclic or part cyclic/acyclic, be unsubstituted, substituted or terminated by one or more substituents selected from halo, cyano, nitro, OR¹⁹, OC(O)R²⁰, C(O)R²¹, C(O)OR²², NR²³R²⁴, C(O)NR²⁵R²⁶, SR²⁹, C(O)SR³⁰, C(S)NR²⁷R²⁸, unsubstituted or substituted aryl, or unsubstituted or substituted Het, wherein R¹⁹ to R³⁰ are defined herein and/or be interrupted by one or more (preferably less than 4) oxygen, sulphur, silicon atoms, or by silano or dialkylsilicon groups, or mixtures thereof.

The term “alkynyl” when used herein, means C₂ to C₁₀ alkynyl and includes ethynyl, propynyl, butynyl, pentynyl, and hexynyl groups. Unless otherwise specified, alkynyl groups may, when there is a sufficient number of carbon atoms, be linear or branched, be saturated or unsaturated, be cyclic, acyclic or part cyclic/acyclic, be unsubstituted, substituted or terminated by one or more substituents selected from halo, cyano, nitro, OR¹⁹, OC(O)R²⁰, C(O)R²¹, C(O)OR²², NR²³R²⁴, C(O)NR²⁵R²⁶, SR²⁹, C(O)SR³⁰, C(S)NR²⁷R²⁸, unsubstituted or substituted aryl, or unsubstituted or substituted Het, wherein R¹⁹ to R³⁰ are defined herein and/or be interrupted by one or more (preferably less than 4) oxygen, sulphur, silicon atoms, or by silano or dialkylsilicon groups, or mixtures thereof.

The terms “alkyl”, “aralkyl”, “alkaryl”, “arylenealkyl” or the like should, in the absence of information to the contrary, be taken to be in accordance with the above definition of “alkyl” as far as the alkyl or alk portion of the group is concerned.

The above Ar or aryl groups may be attached by one or more covalent bonds but references to “arylene” or “arylenealkyl” or the like herein should be understood as two covalent bond attachment but otherwise be defined as Ar or aryl above as far as the arylene portion of the group is concerned. References to “alkaryl”, “aralkyl” or the like should be taken as references to Ar or aryl above as far as the Ar or aryl portion of the group is concerned.

Halo groups with which the above-mentioned groups may be substituted or terminated include fluoro, chloro, bromo and iodo.

The term “Het”, when used herein, includes four- to twelve-membered, preferably four- to ten-membered ring systems, which rings contain one or more heteroatoms selected from nitrogen, oxygen, sulfur and mixtures thereof, and which rings contain no, one or more double bonds or may be non-aromatic, partly aromatic or wholly aromatic in character. The ring systems may be monocyclic, bicyclic or fused. Each “Het” group identified herein may be unsubstituted or substituted by one or more substituents selected from halo, cyano, nitro, oxo, alkyl (which alkyl group may itself be unsubstituted or substituted or terminated as defined herein) —OR¹⁹, —OC(O)R²⁰, —C(O)R²¹, —C(O)OR²², —N(R²³)R²⁴, —C(O)N(R²⁵)R²⁶, —SR²⁹, —C(O)SR³⁰ or —C(S)N(R²⁷)R²⁸ wherein R¹⁹ to R³⁰ are as defined herein The term “Het” thus includes groups such as optionally substituted azetidinyl, pyrrolidinyl, imidazolyl, indolyl, furanyl, oxazolyl, isoxazolyl, oxadiazolyl, thiazolyl, thiadiazolyl, triazolyl, oxatriazolyl, thiatriazolyl, pyridazinyl, morpholinyl, pyrimidinyl, pyrazinyl, quinolinyl, isoquinolinyl, piperidinyl, pyrazolyl and piperazinyl. Substitution at Het may be at a carbon atom of the Het ring or, where appropriate, at one or more of the heteroatoms.

“Het” groups may also be in the form of an N oxide.

The term hetero as mentioned herein means nitrogen, oxygen, sulfur or mixtures thereof.

The adamantyl, congressyl, norbornyl or 1-norborndienyl group may optionally comprise, besides hydrogen atoms, one or more substituents selected from alkyl, —OR¹⁹, —OC(O)R²⁰, halo, nitro, —C(O)R²¹, —C(O)R²², cyano, aryl, —N(R²³)R²⁴, —C(O)N(R²⁵)R²⁶, —C(S)(R²⁷)R²⁸, —SR²⁹, —C(O)SR³, —CF₃, —P(R⁵⁶)R⁵⁷, —PO(R⁵⁸)(R⁵⁹), —PO₃H₂, —PO(OR⁶⁰) (OR⁶¹), or —SO₃R⁶², wherein R¹⁹-R³⁰, alkyl, halo, cyano and aryl are as defined herein and R⁵⁶ to R⁶² each independently represent hydrogen, alkyl, aryl or Het.

Suitably, when the adamantyl, congressyl, norbornyl or 1-norborndienyl group is substituted with one or more substituents as defined above, highly preferred substituents include unsubstituted C₁ to C₈ alkyl, —OR¹⁹, —OC(O)R²⁰, phenyl, —C(O)OR²², fluoro, —SO₃H, —N(R²³)R²⁴, —P(R⁵⁶)R⁵⁷, —C(O)N(R²⁵)R²⁶ and PO(R⁵⁸)(R⁵⁹), —CF₃, wherein R¹⁹ represents hydrogen, unsubstituted C₁-C₈ alkyl or phenyl, R²⁰, R²², R²³, R²⁴, R²⁵, R²⁶ each independently represent hydrogen or unsubstituted C₁-C₈ alkyl, R⁵⁶ to R⁵⁹ each independently represent unsubstituted C₁-C₈ alkyl or phenyl. In a particularly preferred embodiment the substituents are C₁ to C₈ alkyl, more preferably, methyl such as found in 1,3 dimethyl adamantyl.

Suitably, the adamantyl, congressyl, norbornyl or 1-norborndienyl group may comprise, besides hydrogen atoms, up to 10 substituents as defined above, preferably up to 5 substituents as defined above, more preferably up to 3 substituents as defined above. Suitably, when the adamantyl, congressyl, norbornyl or 1-norborndienyl group comprises, besides hydrogen atoms, one or more substituents as defined herein, preferably each substituent is identical. Preferred substituents are unsubstituted C₁-C₈ alkyl and trifluoromethyl, particularly unsubstituted C₁-C₈ alkyl such as methyl. A highly preferred adamantyl, congressyl, norbornyl or 1-norborndienyl group comprises hydrogen atoms only i.e. the adamantyl congressyl, norbornyl or 1-norborndienyl group is not substituted.

Preferably, when more than one adamantyl, congressyl, norbornyl or 1-norborndienyl group is present in a compound of formula I, each such group is identical.

Preferably, the bidentate ligand is a bidentate phosphine, arsine or stibine ligand, preferably, a bidentate phosphine ligand.

For the avoidance of doubt, references to Group 8, 9 or 10 metals herein should be taken to include Groups 8, 9 and 10 in the modern periodic table nomenclature. By the term “Group 8, 9 or 10” we preferably select metals such as Ru, Rh, Os, Ir, Pt and Pd. Preferably, the metals are selected from Ru, Pt and Pd. More preferably, the metal is Pd.

Suitable compounds of such Group 8, 9 or 10 metals include salts of such metals with, or compounds comprising weakly coordinated anions derived from, nitric acid; sulphuric acid; lower alkanoic (up to C₁₂) acids such as acetic acid and propionic acid; sulphonic acids such as methane sulphonic acid, chlorosulphonic acid, fluorosulphonic acid, trifluoromethane sulphonic acid, benzene sulphonic acid, naphthalene sulphonic acid, toluene sulphonic acid, e.g. p-toluene sulphonic acid, t-butyl sulphonic acid, and 2-hydroxypropane sulphonic acid; sulphonated ion exchange resins (including low acid level sulphonic resins) perhalic acid such as perchloric acid; halogenated carboxylic acids such as trichloroacetic acid and trifluoroacetic acid; orthophosphoric acid; phosphonic acids such as benzenephosphonic acid; and acids derived from interactions between Lewis acids and Broensted acids. Other sources which may provide suitable anions include the optionally halogenated tetraphenyl borate derivatives, e.g. perfluorotetraphenyl borate. Additionally, zero valent palladium complexes particularly those with labile ligands, e.g. triphenylphosphine or alkenes such as dibenzylideneacetone or styrene or tri(dibenzylideneacetone)dipalladium may be used.

The above anions may be introduced directly as a compound of the metal but may also be introduced to the catalyst system independently of the metal or metal compound. Preferably, they are introduced as the acid. Preferably, an acid is selected to have a pKa in the same range as that for the carboxylic acids and aromatic carboxylic acids listed supra

The pKa of the acid is preferably greater than about 2 measured in dilute aqueous solution at 18° C. The pKa is preferably less than about 6 measured in dilute aqueous solution at 18° C. Suitable acids and salts may be selected from the acids and salts listed supra.

Particularly preferred anions for the carbonylation reaction such as a hydroxycarbonylation are the carboxylic acids and aromatic carboxylic acids listed supra. There may be a mixture of anions but preferably only one source of anions is added to the process. However, it should be appreciated that a further source of anions is generated by the process ie the acid product of the carbonylation, for instance pentenoic acid in the carbonylation of 1,3-butadiene.

In the carbonylation reaction such as a hydroxycarbonylation reaction, the quantity of anion present is not critical to the catalytic behaviour of the catalyst system. The molar ratio of anion to Group 8, 9 or 10 metal/compound may be from 1:1 to 10⁷:1, preferably from 2:1 to 10⁷:1 most preferably, from 100:1 to 10⁵:1 and especially 100:1 and 1000:1. Where the anion is provided by an acid and salt, the relative proportion of the acid and salt is not critical Accordingly, if a co-reactant should react with the acid serving as source of anions, then the amount of the acid to co-reactant should be chosen such that a suitable amount of free acid is present.

As mentioned, the catalyst system of the present invention may be used homogeneously or heterogeneously. Preferably, the catalyst system is used homogeneously.

The process of the present invention is particularly efficacious for the production of pentenoic acid isomers. Adipic acid may be produced from pentenoic acid isomers by further carbonylation of the pentenoic acid. Adipic acid is advantageously used as a starting compound in the production of Nylon 6,6.

Therefore, the invention also relates specifically to the carbonylation reaction such as a hydroxycarbonylation of conjugated diene, especially 1,3-butadiene, and, in particular but not exclusively, the use of the carbonylation reaction such as a hydroxycarbonylation to provide a first step in the production of adipic acid from pentenoic acid such as 3-pentenoic acid.

Accordingly, in a third aspect of the present invention there is provided a process for the production of isomers of pentenoic acid, the said process comprising the steps of hydroxycarbonylating 1,3-butadiene according to the first or second aspect of the invention.

Therefore, according to a fourth aspect of the present invention there is provided a process for the production of adipic acid, said process comprising the steps of hydroxycarbonylating 1,3-butadiene in accordance with the first or second aspect of the present invention to produce a product comprising an isomer(s) of pentenoic acid which may be substituted or unsubstituted and either branched or linear and treatment of the said pentenoic acid product to produce adipic acid.

Alternatively, depending on the co-reactant other reactions can be carried out followed by further treatment. By treating or treatment herein is meant carrying out routine chemical treatment such as carbonylation on the product of the carbonylation reaction to produce adipic acid in the case of hydroxycarbonylation or hexamethylenediamine, or ε-caprolactam in the case of ammonia or an amide respectively.

According to a fifth aspect of the present invention there is provided the use of the catalyst system as defined in any of the aspects of the present invention for the production, preferably, industrial production, of adipic acid, the said production comprising the steps of hydroxycarbonylation of 1,3-butadiene followed by treatment of the pentenoic acid product of the hydroxycarbonylation to produce the adipic acid.

Preferably, the treatment as mentioned above is carbonylation. A suitable process for the carbonylation of pentenoic acid is described in WO0248094A1 where 3-pentenoic acid is hydroxycarbonylated.

The product adipic acid may be used in the preparation of Nylon 6,6.

For ease of reference, any one or more of the aspects of the invention may be referred to herein as the process of the invention.

Conveniently, the process of the invention may utilise highly stable compounds under typical carbonylation reaction such as a hydroxycarbonylation reaction conditions such that they require little or no replenishment. Conveniently, the process of the invention may have a high rate for the carbonylation reaction such as a hydroxycarbonylation reaction of conjugated diene. Conveniently, the process of the invention may promote high conversion rates of conjugated diene, thereby yielding the desired product in high yield with little or no impurities. Consequently, the commercial viability of the carbonylation reaction of any conjugated diene may be increased by employing the process of the invention. Especially advantageously, the process of the invention allows for a carbonylation reaction such as a hydroxycarbonylation reaction with a high TON number and a high rate of reaction.

It will be appreciated by those skilled in the art that the compounds of formula (I) may function as ligands that coordinate with the Group 8, 9 or 10 metal or compound thereof to form the compounds for use in the invention. Typically, the Group 8, 9 or 10 metal or compound thereof coordinates to the one or more phosphorus, arsenic and/or antimony atoms of the compound of formula (I).

The present invention provides a process for the carbonylation reaction such as a hydroxycarbonylation of conjugated dienes comprising contacting a conjugated diene with carbon monoxide in the presence of a solvent system comprising an aromatic carboxylic acid, a catalyst and, optionally, a source of hydrogen as defined in the present invention.

The catalyst compounds of the present invention may act as a “heterogeneous” catalyst or a “homogeneous” catalyst, preferably, a homogenous catalyst.

By the term “homogeneous” catalyst we mean a catalyst, i.e. a compound of the invention, which is not supported but is simply admixed or formed in-situ with the reactants of the carbonylation reaction, preferably in a suitable co-solvent as described herein.

By the term “heterogeneous” catalyst we mean a catalyst, i.e. the compound of the invention, which is carried on a support.

Thus according to a further aspect, the present invention provides a process for the carbonylation of a conjugated diene as defined herein wherein the process is carried out with the catalyst comprising a support, preferably an insoluble support.

Preferably, the support comprises a polymer such as a polyolefin, polystyrene or polystyrene copolymer such as a divinylbenzene copolymer or other suitable polymers or copolymers known to those skilled in the art; a silicon derivative such as a functionalised silica, a silicone or a silicone rubber; or other porous particulate material such as for example inorganic oxides and inorganic chlorides.

Preferably the support material is porous silica which has a surface area in the range of from 10 to 700 m²/g, a total pore volume in the range of from 0.1 to 4.0 cc/g and an average particle size in the range of from 10 to 500 μm. More preferably, the surface area is in the range of from 50 to 500 m²/g, the pore volume is in the range of from 0.5 to 2.5 cc/g and the average particle size is in the range of from 20 to 200 μm. Most desirably the surface area is in the range of from 100 to 400 m²/g, the pore volume is in the range of from 0.8 to 3.0 cc/g and the average particle size is in the range of from 30 to 100 μm. The average pore size of typical porous support materials is in the range of from 10 to 1000 Å. Preferably, a support material is used that has an average pore diameter of from 50 to 500 Å, and most desirably from 75 to 350 Å. It may be particularly desirable to dehydrate the silica at a temperature of from 100° C. to 800° C. anywhere from 3 to 24 hours.

Suitably, the support may be flexible or a rigid support, the insoluble support is coated and/or impregnated with the compounds of the process of the invention by techniques well known to those skilled in the art.

Alternatively, the compounds of the process of the invention are fixed to the surface of an insoluble support, optionally via a covalent bond, and the arrangement optionally includes a bifunctional spacer molecule to space the compound from the insoluble support.

The compounds of the invention may be fixed to the surface of the insoluble support by promoting reaction of a functional group present in the compound of formula I with a complimentary reactive group present on or previously inserted into the support. The combination of the reactive group of the support with a complimentary substituent of the compound of the invention provides a heterogeneous catalyst where the compound of the invention and the support are linked via a linkage such as an ether, ester, amide, amine, urea, keto group.

The choice of reaction conditions to link a compound of the process of the present invention to the support depends upon the groups of the support. For example, reagents such as carbodiimides, 1,1-carbonyldiimidazole, and processes such as the use of mixed anhydrides, reductive amination may be employed.

According to a further aspect, the present invention provides the use of the process or catalyst of any aspect of the invention wherein the catalyst is attached to a support.

Additionally, the bidentate ligand may be bonded to a suitable polymeric substrate via at least one of the bridge substituents (including the cyclic atoms) the bridging group X, the linking group L¹ or the linking group L² e.g. cis-1,2-bis(di-t-butylphosphinomethyl)benzene may be bonded, preferably, via the 3, 4, 5 or 6 cyclic carbons of the benzene group to polystyrene to give an immobile heterogeneous catalyst.

Suitably, the catalysts of the invention are prepared in a separate step preceding their use in-situ in the carbonylation reaction.

Conveniently, the process of the invention may be carried out by dissolving the Group 8, 9 or 10 metal or compound thereof as defined herein in a suitable solvent such as one of the alkanols or aprotic solvents previously described (a particularly preferred solvent would be the product of the specific carbonylation reaction and subsequently admixing with a compound of formula I as defined herein.

The carbon monoxide may be used in the presence of other gases which are inert in the reaction. Examples of such gases include hydrogen, nitrogen, carbon dioxide and the noble gases such as argon.

The product of the reaction may be separated from the other components by any suitable means. However, it is an advantage of the present process that significantly fewer by-products are formed thereby reducing the need for further purification after the initial separation of the product as may be evidenced by the generally significantly higher selectivity. A further advantage is that the other components which contain the catalyst system which may be recycled and/or reused in further reactions with minimal supplementation of fresh catalyst.

There is no particular restriction on the duration of the carbonylation except that carbonylation in a timescale which is commercially acceptable is obviously preferred. Carbonylation in a batch reaction may take place in up to 48 hours, more typically, in up to 24 hours and most typically in up to 12 hours. Typically, carbonylation is for at least 5 minutes, more typically, at least 30 minutes, most typically, at least 1 hour. In a continuous reaction such time scales are obviously irrelevant and a continuous reaction can continue as long as the TON is commercially acceptable before catalyst requires replenishment.

The catalyst system of the present invention is preferably constituted in the liquid phase which may be formed by one or more of the reactants or by the use of the solvent.

The use of stabilising compounds with the catalyst system may also be beneficial in improving recovery of metal which has been lost from the catalyst system. When the catalyst system is utilized in a liquid reaction medium such stabilizing compounds may assist recovery of the group 8, 9 or 10 metal.

Preferably, therefore, the catalyst system includes in a liquid reaction medium a polymeric dispersant dissolved in a liquid carrier, said polymeric dispersant being capable of stabilising a colloidal suspension of particles of the group 8, 9 or 10 metal or metal compound of the catalyst system within the liquid carrier.

The liquid reaction medium may be a solvent for the reaction or may comprise one or more of the reactants or reaction products themselves. The reactants and reaction products in liquid form may be miscible with or dissolved in a solvent or liquid diluent.

The polymeric dispersant is soluble in the liquid reaction medium, but should not significantly increase the viscosity of the reaction medium in a way which would be detrimental to reaction kinetics or heat transfer. The solubility of the dispersant in the liquid medium under the reaction conditions of temperature and pressure should not be so great as to deter significantly the adsorption of the dispersant molecules onto the metal particles.

The polymeric dispersant is capable of stabilising a colloidal suspension of particles of said group 8, 9 or 10 metal or metal compound within the liquid reaction medium such that the metal particles formed as a result of catalyst degradation are held in suspension in the liquid reaction medium and are discharged from the reactor along with the liquid for reclamation and optionally for re-use in making further quantities of catalyst. The metal particles are normally of colloidal dimensions, e.g. in the range 5-100 nm average particle size although larger particles may form in some cases. Portions of the polymeric dispersant are adsorbed onto the surface of the metal particles whilst the remainder of the dispersant molecules remain at least partially solvated by the liquid reaction medium and in this way the dispersed group 8, 9 or 10 metal particles are stabilised against settling on the walls of the reactor or in reactor dead spaces and against forming agglomerates of metal particles which may grow by collision of particles and eventually coagulate. Some agglomeration of particles may occur even in the presence of a suitable dispersant but when the dispersant type and concentration is optimised then such agglomeration should be at a relatively low level and the agglomerates may form only loosely so that they may be broken up and the particles redispersed by agitation.

The polymeric dispersant may include homopolymers or copolymers including polymers such as graft copolymers and star polymers.

Preferably, the polymeric dispersant has sufficiently acidic or basic functionality to substantially stabilise the colloidal suspension of said group 8, 9 or 10 metal or metal compound.

By substantially stabilise is meant that the precipitation of the group 8, 9 or 10 metal from the solution phase is substantially avoided.

Particularly preferred dispersants for this purpose include acidic or basic polymers including carboxylic acids, sulphonic acids, amines and amides such as polyacrylates or heterocycle, particularly nitrogen heterocycle, substituted polyvinyl polymers such as polyvinyl pyrrolidone or copolymers of the aforesaid.

Examples of such polymeric dispersants may be selected from polyvinylpyrrolidone, polyacrylamide, polyacrylonitrile, polyethylenimine, polyglycine, polyacrylic acid, polymethacrylic acid, poly(3-hydroxybutyricacid), poly-L-leucine, poly-L-methionine, poly-L-proline, poly-L-serine, poly-L-tyrosine, poly(vinylbenzenesulphonic acid) and poly(vinylsulphonic acid), acylated polyethylenimine. Suitable acylated polyethylenimines are described in BASF patent publication EP1330309 A1 and U.S. Pat. No. 6,723,882.

Preferably, the polymeric dispersant incorporates acidic or basic moieties either pendant or within the polymer backbone. Preferably, the acidic moieties have a dissociation constant (pKa) of less than 6.0, more preferably, less than 5.0, most preferably less than 4.5. Preferably, the basic moieties have a base dissociation constant (pK_{b}) being of less than 6.0, more preferably less than 5.0 and most preferably less than 4.5, pKa and pK_{b} being measured in dilute aqueous solution at 25° C.

Suitable polymeric dispersants, in addition to being soluble in the reaction medium at reaction conditions, contain at least one acidic or basic moiety, either within the polymer backbone or as a pendant group. We have found that polymers incorporating acid and amide moieties such as polyvinylpyrollidone (PVP) and polyacrylates such as polyacrylic acid (PAA) are particularly suitable. The molecular weight of the polymer which is suitable for use in the invention depends upon the nature of the reaction medium and the solubility of the polymer therein. We have found that normally the average molecular weight is less than 100,000. Preferably, the average molecular weight is in the range 1,000-200,000, more preferably, 5,000-100,000, most preferably, 10,000-40,000 e.g. Mw is preferably in the range 10,000-80,000, more preferably 20,000-60,000 when PVP is used and of the order of 1,000-10,000 in the case of PAA.

The effective concentration of the dispersant within the reaction medium should be determined for each reaction/catalyst system which is to be used.

The dispersed group 8, 9 or 10 metal may be recovered from the liquid stream removed from the reactor e.g. by filtration and then either disposed of or processed for re-use as a catalyst or other applications. In a continuous process the liquid stream may be circulated through an external heat-exchanger and in such cases it may be convenient to locate filters for the palladium particles in these circulation apparatus.

Preferably, the polymer:metal mass ratio in g/g is between 1:1 and 1000:1, more preferably, between 1:1 and 400:1, most preferably, between 1:1 and 200:1. Preferably, the polymer:metal mass ratio in g/g is up to 1000, more preferably, up to 400, most preferably, up to 200.

It will be appreciated that any of the features set forth in the first aspect of the invention may be regarded as preferred features of the second, third or other aspect of the present invention and vice versa.

The invention also extends to novel bidentate ligands of formula (I) and novel complexes of such ligands with the metal of Group 8, 9 or 10 or a compound thereof.

The invention will now be described and illustrated by way of the following non-limiting examples and comparative examples.

### Carbonylation Experimental Procedure

Reaction solutions were prepared using standard Schlenk line techniques. 112.5 mg (0.500 mmoles) of Pd(OAc)₂ and 494 mg (1.25 mmoles) of the bidentate ligand 1,2-bis(di-tert-butylphosphinomethyl)benzene were weighed into a round-bottom flask in a nitrogen purge glovebox. The round-bottom flask was then transferred to a Schlenk line. 100 ml of degassed solvent (as defined in the following examples), 100 ml of degassed carboxylic acid (as defined in the following examples) (if the carboxylic acid is a liquid) and 25 ml of degassed demineralised water were added to yield a two-phase solution, of which the upper, yellow, organic-rich phase contained the catalyst. If the carboxylic acid (as defined in the following examples) was a solid, it was added to the autoclave as a solid (as defined below).

The two-phase catalyst solution was added to an autoclave by suction from the round bottom flask and approximately 100 grams of 1,3-butadiene was added to the catalyst solution by suction from a 300 ml Whitey pressure vessel. The autoclave was heated to 135° C. The reaction was started by the introduction of 40 bar CO into the autoclave from a main-line gas supply, followed by immediately connecting the reaction autoclave to a 2.25 litre feed reservoir vessel containing CO. The autoclave pressure was held constant by maintaining a CO feed from the reservoir to top up the reacted gas. After 3 hours the CO feed was isolated and the autoclave cooled before the pressure was vented. The liquid volume was left within the extracted autoclave overnight to allow the dissolved unreacted 1,3-butadiene to de-gas and vent before being collected for analysis.

Initial reaction rates and turnover numbers (TON) were calculated from the rate of change of pressure in the 2.25 litre CO feed reservoir assuming ideal gas behaviour and 100% selectivity to pentenoic acid formation.

### EXAMPLES 1-4 AND Comparative Examples 1-5

### Variation of Carboxylic Acid

The procedure outlined above was carried out with various carboxylic acids. All reactions were run with 100 ml of toluene (solvent), palladium to bidentate ligand ratio of 1:2.5 and the same number of moles of carboxylic acid as there are in 100 ml of nonanoic acid i.e 0.573 moles (unless otherwise stated). Example 2 was run with the same number of moles of acid as there are in 50 ml of nonanoic acid. The results are detailed in table 1 below.

TABLE 1

| | Acid (number of moles | | |
|---|---|---|---|
| | of acid used is equal | | |
| | to that in 100 ml of | | Max rate |
| Example | nonanoic acid) | TON number | (mol/mol/hr) |
| 1 | O-toluic acid | 385 | 400 |
| 2 | O-toluic acid | 795 | 500 |
| | (equivalent to 50 ml of | | |
| | nonanoic acid | | |
| Comp 1 | Nonanoic acid | 146 | 200 |
| Comp 2 | Lactic acid | 52 | 31 |
| Comp 3 | Succinic acid | 0 | 0 |
| 3 | 3-phenyl propionic | 233 | 146 |
| | acid | | |
| Comp 4 | Malonic acid | decomp | decomp |
| Comp 5 | Pivalic acid | 191 | 147 |
| 4 | Diphenyl acetic | 476 | 359 |
| | acid | | |

### EXAMPLES 5-9 AND Comparative Example 6

### Effect of Altering the Carboxylic Acid in the Reaction Under Partially Optimized Conditions

The method of example 1 was carried out with various carboxylic acids under the partially optimized conditions of a palladium to bidentate ligand ratio of 1:5 but using the same amount of Palladium as the previous examples The results are detailed in table 2 below.

TABLE 2

| | Acid (number of moles | | |
|---|---|---|---|
| | of acid used is equal | | |
| | to that in 50 ml of | | |
| Example | nonanoic acid) | TON number | Max rate |
| 5 | O-toluic acid | 1257 | 656 |
| 6 | Benzoic acid | 1067 | 692 |
| 7 | 2,4,6-trimethyl | 1000 | 917 |
| | benzoic acid | | |
| 8 | Benzilic acid | 179 | 58 |
| 9 | Phenoxy acetic acid | 151 | 244 |
| Comp 6 | Pyruvic acid | 0 | 0 |

### EXAMPLES 10-13

### Variation of Co-Solvent

The method of example 1 was carried out using o-toluic acid as the carboxylic acid, and with various co-solvents (100 ml). The reactions were again carried out in the presence of a palladium to bidentate ligand ratio of 1:5 as in the previous examples. Changing the co-solvent was done in two parts; one where the co-solvent used was miscible with water and the second where the co-solvent used was immiscible with water. The use of dioxane, acetonitrile, and THF co-solvents gave a mono-phasic solution when the bidentate ligand, Palladium acetate, co-solvent and water were added together. The use of Toluene and Methyl-tert-butyl ether (MTBE) gave a bi-phasic solution when the bidentate ligand, palladium acetate, co-solvent and water were added together. The results are detailed in table 3 below.

TABLE 3

| Example | Co-Solvent | TON number | Max rate |
|---|---|---|---|
| 10 | Dioxane | 1073 | 414 |
| 11 | Toluene | 1257 | 656 |
| 12 | MTBE | 742 | 880 |
| 13 | THF | 765 | 670 |

Key: MTBE = methyl-tert-butyl ether
THF = tetrahydrofuran

The mono-phasic solutions were observed to give reasonable TON (in the case of dioxane) and reasonable maximum rate (in the case of THF). The bi-phasic solutions were observed to give high TON (in the case of toluene) and high maximum rate (in the case of MTBE).

### EXAMPLES 14-16

### Variation of Bidentate Ligand to Palladium Ratio

The method of example 1 was carried out using o-toluic acid as the carboxylic acid. All the reactions were run with 100 ml of toluene as co-solvent. The ratio of bidentate ligand to palladium was varied as outlined in table 4 below but now based on 0.125 mmoles Pd(OAc)₂).

TABLE 4

| | Acid (number of moles | | | |
|---|---|---|---|---|
| | of acid used is equal | Equivalent | | |
| | to that in 50 ml of | Alpha of | TON | Maximum |
| Example | nonanoic acid) | ligand | number | rate |
| 14 | O-toluic acid | 10 | — | 1173 |
| 15 | O-toluic acid | 40 | — | 1613 |
| 16 | O-toluic acid | 80 | — | 2648 |

### EXAMPLES 17-21

### Addition of 5 Bar of Hydrogen

The method of example 1 was carried out with o-toluic acid and various palladium to bidentate ligand ratios. However, when hydrogen was used in the reaction the autoclave and its contents were heated to 110° C. When the temperature reached 110° C. the heater and stirrer were stopped and a partial pressure of 5 bar of hydrogen gas was added from an external bomb. After the hydrogen had been added, the heater and stirrer were restarted and the reaction allowed to proceed as usual. The results are shown in table 5 below.

TABLE 5

| | | | Equivalents | | |
|---|---|---|---|---|---|
| | | Hydrogen | of Alpha | TON | |
| Example | Acid | Added | ligand | number | Max rate |
| 17 | O-toluic | Yes | 5 | 1301 | 1066 |
| | acid | | | | |
| 18 | O-toluic | No | 5 | 1257 | 656 |
| | acid | | | | |
| 19 | O-toluic | Yes | 10 | 1011 | 1289 |
| | acid | | | | |
| 20 | O-toluic | Yes | 10 | 1207 | 1341 |
| | acid | | | | |
| 21 | O-toluic | No | 10 | 1419 | 745 |
| | acid | | | | |

The reader's attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings), may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

1. A process for the carbonylation of a conjugated diene comprising the steps of reacting said conjugated diene with carbon monoxide and a co-reactant having an active hydrogen in the presence of a solvent system comprising an aromatic carboxylic acid, a catalyst system and, optionally, a source of hydrogen, the catalyst system obtainable by combining:
(a) a metal of Group 8, 9 or 10 or a compound thereof: and
(b) a bidentate ligand of general formula (I)
X¹(X²)-Q²-A-R—B-Q¹-X³(X⁴) (I)
wherein:
A and B each independently represent lower alkylene linking groups;
R represents a cyclic hydrocarbyl structure to which Q¹ and
Q² are linked, via the said linking group, on available adjacent cyclic atoms of the cyclic hydrocarbyl structure;
the groups X¹, X², X³ and X⁴ independently represent univalent radicals of up to 30 atoms having at least one tertiary carbon atom or X¹ and X² and/or X³ and X⁴ together form a bivalent radical of up to 40 atoms having at least two tertiary carbon atoms wherein each said univalent or bivalent radical is joined via said at least one or two tertiary carbon atoms respectively to the appropriate atom Q¹ or Q²;
Q¹ and Q² each independently represent phosphorus, arsenic or antimony; and optionally, a source or further source of anions.

2. A process for the carbonylation of a conjugated diene comprising the steps of reacting said conjugated diene with carbon monoxide and a co-reactant having an active hydrogen in the presence of a solvent system comprising a carboxylic acid, a catalyst system and, optionally, a source of hydrogen, the catalyst system obtainable by combining:
(a) a metal of Group 8, 9 or 10 or a compound thereof: and
(b) a bidentate ligand of general formula (I)
X¹(X²)-Q²-A-R—B-Q¹-X³(X⁴) (I)
wherein:
A and B each independently represent lower alkylene linking groups;
R represents a cyclic hydrocarbyl structure to which Q¹ and Q² are linked, via the said linking group, on available adjacent cyclic atoms of the cyclic hydrocarbyl structure;
the groups X¹, X¹, X² and X⁴ independently represent univalent radicals of up to 30 atoms having at least one tertiary carbon atom or X¹ and X² and/or X³ and X⁴ together form a bivalent radical of up to 40 atoms having at least two tertiary carbon atoms wherein each said univalent or bivalent radical is joined via said at least one or two tertiary carbon atoms respectively to the appropriate atom Q¹ or Q²;
Q¹ and Q² each independently represent phosphorus, arsenic or antimony; and, optionally, a source of anions;
wherein the ratio of bidentate ligand:group 8, 9 or 10 metal is greater than 10:1 (mol:mol) and wherein the catalyst system includes a polymeric dispersant which is soluble in the liquid reaction medium.

3. A process as claimed in claim 1, wherein the co-reactant is selected from water, a carboxylic acid, alcohol, ammonia or an amine, a thiol, or a combination thereof.

4. A process as claimed in claim 1, wherein the conjugated diene is an optionally substituted conjugated diene having from 4 to 22 atoms per molecule.

5. A process as claimed in claim 1, wherein the aromatic carboxylic acid used in the carbonylation reaction is any optionally substituted C₁-C₃₀ aromatic compound such as those based on phenyl, napthyl, cyclopentadienyl anion(s), indenyl, pyridinyl, and pyrollyl groups and having at least one carboxylic acid group associated with the aromatic ring, more preferably any C₁ to C₁₆ aromatic compound having at least one carboxylic acid group.

6. A process as claimed in claim 1, wherein the pKa of the acid is greater than about 2 measured in dilute aqueous solution at 18° C.

7. A process as claimed in claim 1, wherein the pKa of the acid is less than about 6 measured in dilute aqueous solution at 18° C.

8. A process as claimed in claim 1, wherein the carboxylic acid is substituted with one or more of the following: alkyl groups; aryl groups; hydroxy groups; alkoxy groups such as, for example, methoxy; amino groups; or halo groups such as, for example F, Cl, I and Br.

9. A process as claimed in claim 1, wherein, when present, the aromatic ring of the carboxylic acid is mono- or di-substituted.

10. A process as claimed claim 1, wherein the carboxylic acid is selected from the aromatic carboxylic acids of benzoic acids; naphthoic acids; or cyclopentadienyl acids.

11. A process as claimed in claim 1, wherein, when present, the aromatic carboxylic acids are substituted aromatic carboxylic acids.

12. A process as claimed in claim 1, wherein, when present, the aromatic carboxylic acid is selected from C₁-C₄ alkyl substituted benzoic acids, such as 2,4,6-trimethyl benzoic acid, 2,6-dimethyl benzoic acid and O-toluic acid (2-methyl benzoic acid), 2-nitrobenzoic acid, 6-chloro-2-methylolbenzoic acid, 4-aminobenzoic acid, 2-chloro-6-hydroxybenzoic acid, 2-cyanobenzoic acid, 3-cyanobenzoic acid, 4-cyanobenzoic acid 2,4-dihydroxybenzoic, 3-nitrobenzoic acid, 2-phenylbenzoic acid, 2-tert-butylbenzoic acid, 2-napthoic acid, 1-napthoic acid, 2,4-dimethylbenzoic acid, 3-methylbenzoic acid, 3,5-dimethylbenzoic acid, 4-hydroxybenzoic acid, 2-fluorobenzoic acid, 3-propoxybenzoic acid, 3-ethoxybenzoic acid, 2-propoxybenzoic acid, 2,2-diphenylpropionic acid, 2-methoxyphenylacetic acid, ortho-anisic acid, meta-anisic acid, 4-tert-butylbenzoic acid and 2-ethoxybenzoic acid.

13. A process as claimed in claim 1, wherein, when present, the aromatic carboxylic acid is substituted by only one group in addition to the group bearing the carboxylic acid.

14. A process according to claim 2, wherein the carboxylic acid is any optionally substituted C₁-C₃₀ compound having, in addition, at least one carboxylic acid group.

15. A process according to claim 2, wherein the carboxylic acid is the acid product of the carbonylation reaction.

16. A process according to claim 1, wherein the solvent system comprises in addition to a carboxylic acid as defined above, at least one co-solvent.
